(19) United States
(12) Patent Application Publication
(10) Pub. No.: US 2016/0058796 A1
(43) Pub. Date: Mar. 3, 2016

(54) RETINA EXTRACELLULAR MATRIX BASED BIOMATERIAL

(71) Applicant: NORTHEASTERN UNIVERSITY, Boston, MA (US)

(72) Inventors: Rebecca Lyn Carrier, Needham, MA (US); Joydip Kundu, Malden, MA (US)

(21) Appl. No.: 14/787,359

(22) PCT Filed: May 5, 2014
(86) PCT No.: PCT/US14/36804

§ 371 Date: Oct. 27, 2015

Related U.S. Application Data
(60) Provisional application No. 61/819,395, filed on May 3, 2013.

(51) Int. Cl. A61K 35/30; A61K 47/46; C12N 5/079; A61K 9/00

(52) U.S. Cl. CPC A61K 35/30; A61K 9/0051; A61K 47/46; C12N 5/0621; C12N 2533/90

(57) ABSTRACT

The present technology relates to compositions and methods useful for preventing and treating degenerative eye diseases or disorders. The compositions and methods include ocular biomaterials. In some embodiments, the ocular biomaterial compositions include retinal biomaterial, interphotoreceptor matrix biomaterial or a combination thereof. In some embodiments, the ocular biomaterial compositions are formed into films, gels, scaffolds and matrices for cell delivery.

### CROSS-REFERENCE TO RELATED APPLICATIONS

This application claims priority to U.S. Application No. 61/819,395 filed May 3, 2013, the entire contents of which is hereby incorporated by reference in its entirety.

### GOVERNMENT SUPPORT

This invention was made with U.S. Government support under grant 1R21EY021312 awarded by the National Institutes of Health. The U.S. Government has certain rights in the invention.

### BACKGROUND

The following description is provided to assist the understanding of the reader. None of the information provided or references cited is admitted to be prior art to the present technology.

Retinal degenerative diseases, such age-related macular degeneration (AMD), impact millions of people. Retinal degeneration and associated photoreceptor loss is the leading cause of blindness in the industrialized world. It is estimated that 25 to 30 million people worldwide suffer from AMD. See e.g., Vugler et al., Mechanisms of development, 124(11-12): 807-829 (2007). Current treatment strategies, including gene therapy, anti-angiogenic therapy, and growth factor treatment, focus mainly on slowing the degeneration process.

### SUMMARY

In one aspect, the present technology provides for a composition including a decellularized interphotoreceptor matrix (IPM)-based biomaterial. In some embodiments, the composition also includes a decellularized retina-based biomaterial. In some embodiments, the composition is substantially free of one or more biological inhibitory compounds selected from the group consisting of: chondroitin sulfate, proteoglycans, and leukocyte antigens.

In some embodiments, the composition also includes one or more biological agents, synthetic agents, or a combination thereof, wherein the biological agent and/or synthetic agent is dispersed throughout the decellularized IPM-based biomaterial. In some embodiments, the biological agent is one or more selected from the group consisting of collagen, fibronectin, and transglutaminase. In some embodiments, the synthetic agent is one or more selected from the group consisting of polymers, poly(lactic-co-glycolic acid) (PLGA), polyglycerol sebacate (PGS), poly(L-lactic acid) (PLLA), poly(methylmethacrylate) (PMMA), and polycaprolactone (PCL).

In some embodiments, the composition also includes one or more additional components, wherein the additional components are selected from the group consisting of laminin, an antibiotic, an antiviral, an antifungal, growth factors, cytokines/chemokines, hormones, cell signaling molecules, a gelling agent, a protease inhibitor, cell media, and bovine serum proteins.

In some embodiments, the decellularized IPM-based biomaterial is derived from a mammal, an amphibian, a fish, or a combination thereof. In some embodiments, the decellularized IPM-based biomaterial is derived from a species in which retinal regeneration occurs.

In some embodiments, the composition also includes retinal progenitor epithelium cells (RPCs). In some embodiments, the RPCs are human.

In some embodiments, the composition is applied onto a substrate, wherein the substrate is selected from the group consisting of a biological or polymer membrane, tissue culture inserts, and cell culture plates. In some embodiments, the composition is formed as a cell scaffold.

In another aspect, the present technology provides methods for preventing or treating retinal degeneration in a subject in need thereof comprising. The methods include combining retinal cells and/or retinal progenitor cells (RPCs) with any of the ocular biomaterial compositions disclosed herein to form a cell delivery matrix and implanting the cell delivery matrix in at least one eye of the subject.

In some embodiments, the retinal degeneration is caused by a disease or disorder selected from the group consisting of: macular degeneration, retinitis pigmentosa, diabetic retinopathy, hypertensive retinopathy, radiation retinopathy, solar retinopathy, retinopathy of and prematurity.

In some embodiments, the cell delivery matrix is implanted in the subretinal space of the eye. In some embodiments, the subject is human.

In another aspect, the present technology provides methods for restoring vision to a subject in need thereof. The methods include combining retinal cells and/or retinal progenitor cells (RPCs) with any of the ocular biomaterial compositions disclosed herein to form a cell delivery matrix and implanting the cell delivery matrix in at least one eye of the subject.

In some embodiments, the cell delivery matrix is implanted in the subretinal space of the eye.

In some embodiments, the subject suffers from a disease or disorder selected from the group consisting of: macular degeneration, retinitis pigmentosa macular, diabetic retinopathy, hypertensive retinopathy, radiation retinopathy, solar retinopathy, and retinopathy of prematurity.

In another aspect, the present technology provides for kits. In some embodiments the kit includes at least one ocular biomaterial composition. In some embodiments, the ocular biomaterial composition is a retina-based biomaterial composition, an IPM-based biomaterial composition, or a combination thereof.

In some embodiments, the kit also includes media, solutions, or reagents for combining the ocular biomaterial composition to cells to form a cell delivery matrix. In some embodiments, the kit also includes cells. Cells include, but are not limited to, retinal cells, retinal progenitor cells, retinal stem cells, embryonic stem cells (ESCs), retinal pigment epithelium, glial cells, and stem cells. In some embodiments, the kit includes instructions for combining the ocular biomaterial matrix and cells to form the cell delivery matrix.

In some embodiments, the kit includes a tool or tools for implanting or delivering cell delivery matrix. In some embodiments, the kit also includes directions for implanting or delivering the cell delivery matrix.

### BRIEF DESCRIPTION OF DRAWINGS

FIGS. 1A-C is a schematic representation of the dissection and isolation of bovine retina.

FIG. 2A-D are images that show the surface characterization and topology of the cast dried decellularized retina-based biomaterial matrix. Phase contrast light microscope and scanning electron microscope (SEM) images of decellularized retina-based biomaterial matrix film (A and B) and ethanol treated decellularized retina-based biomaterial matrix (C and D) respectively. The inset image shows the magnified view of the surfaces.

FIG. 3A is a graph showing the concentration of sulfated GAGs (μg/mg of the dry retina) in decellularized, isolated retina-based biomaterial matrix. Differences were considered statistically significant at *P<0.05 and **P<0.001.

FIG. 3B is a graph showing the concentration of hyaluronic acid (HA) (μg/mg of the dry retina) in decellularized retina-based biomaterial matrix. Differences were considered statistically significant at *P<0.05 and **P<0.001.

FIG. 3C is a graph showing the concentration of collagen (μg/mg of the dry retina) in decellularized, isolated retina-based biomaterial matrix. Differences were considered statistically significant at *P<0.05 and **P<0.001.

FIG. 3D is a graph showing the concentration of host DNA (μg/mg of the dry retina) in decellularized, isolated retina-based biomaterial matrix. Differences were considered statistically significant at *P<0.05 and **P<0.001.

FIG. 4A is a graph showing basic fibroblastic growth factor (bFGF) concentration (ng/mg of the dry retina) in urea-heparin extracts of decellularized, isolated retina-based biomaterial matrix from bovine eyes.

FIG. 4B is a graph showing epidermal growth factor (EGF) concentration (ng/mg of the dry retina) in urea-heparin extracts of decellularized, isolated retina-based biomaterial matrix from bovine eyes.

FIG. 4C is a graph showing nerve growth factor (NGF) concentration (ng/mg of the dry retina) in urea-heparin extracts of decellularized, isolated retina-based biomaterial matrix from bovine eyes.

FIGS. 5A-F are stained culture plates showing F-Actin staining of human retinal progenitor cells (hRPCs) plated onto tissue culture plate surface (TCPS), decellularized, isolated retina-based biomaterial matrix film casted on TCPS, and fibronectin coated onto TCPS after day 1 (FIG. 5A-C, respectively) and day 7 (FIG. 5D-F, respectively) of culture.

FIG. 6 is a gel showing the reverse transcriptase-polymerase chain reaction (RT-PCR) detection of retinal outer membrane-1 (ROM), Rhodopsin, Cone-rod homeobox (CRX), neural retina leucine zipper (NRL), and β-actin (housekeeping gene) on D0 Fn (day 0, fibronectin coated on TCPS), D1 Fn (day 1, fibronectin coated on TCPS), D1 Retina (day 1, decellularized retina-based biomaterial matrix film casted on TCPS), D1 TCPS (day 1, on TCPS), D7 Fn (day 7, fibronectin coated on TCPS), D7 Retina (day 7, decellularized retina-based biomaterial matrix film casted on TCPS), D7 TCPS (day 7, on TCPS).

FIGS. 7A-F are stained culture plates showing Live/Dead assay of hRPCs viability on tissue culture plate surface (TCPS), decellularized, isolated retina-based biomaterial matrix film casted on TCPS and fibronectin coated on TCPS after day 1 (FIG. 7A-C, respectively) and day 7 (FIG. 7D-F, respectively) of culture.

FIG. 8 is a graph showing hRPCs proliferation on tissue culture plate surface (TCPS), decellularized, isolated retina-based biomaterial matrix film casted on TCPS, and fibronectin coated on TCPS after day 1 and day 7 of culture. *=P<0.05.

FIG. 9A is a photograph of isolated native interphotoreceptor matrix (IPM) sheets in de-ionized water.

FIG. 9B is a phase contrast light microscopy images of native IPM sheets.

FIG. 9C is an image showing lectin staining by Fluorescein labeled Peanut Agglutinin (FPNA) of native IPM, i.e., IPM that was collected after removal of retinas and any retina debris. at 4696×g for 20 minutes at 25° C.

FIG. 9D is an image showing lectin staining by Fluorescein labeled Peanut Agglutinin (FPNA) of pre-centrifuged IPM, i.e., concentrated IPM prepared via centrifugation of the native IPM suspension.

FIG. 9E is a SEM image of native IPM.

FIG. 9F is a SEM image of pre-centrifuged IPM.

FIGS. 10A-T are images of Live/Dead assay showing hRPCs viability on control (FIGS. 10A-D); native IPM (FIGS. 10E-F); native IPM+chondroitinase (FIGS. 10I-L); pre-centrifuged IPM (FIGS. 10M-P), and pre-centrifuged IPM+chondroitinase (FIGS. 10Q-T) after day 1 and day 7 of culture.

FIGS. 11A-J are images of F-actin staining showing hRPCs morphology on control (FIGS. 11A and F); native IPM (FIGS. 11B and G); native IPM+chondroitinase (FIGS. 11C and H); pre-centrifuged IPM (FIGS. 11D and I), and pre-centrifuged IPM+chondroitinase (FIGS. 11E and J) after day 1 and day 7 of culture.

FIG. 12 is a graph showing hRPCs proliferation on control; native IPM; native IPM+chondroitinase; pre-centrifuged IPM, and pre-centrifuged IPM+chondroitinase after day 1 and day 7 of culture. Differences were considered statistically significant at *P<0.05 and **P<0.001.

FIGS. 13A-DD are images showing differentiation of hRPCs by immunocytochemistry; control (FIGS. 13A-F); native IPM (FIGS. 13G-L); native IPM+chondroitinase (FIGS. 13M-R); pre-centrifuged IPM (FIGS. 13S-X); and pre-centrifuged IPM+chondroitinase (FIGS. 13Y-DD) after day 1 of culture.

### DETAILED DESCRIPTION

It is to be appreciated that certain aspects, modes, embodiments, variations and features of the invention are described below in various levels of detail in order to provide a substantial understanding of the present invention. The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural references unless the content clearly dictates otherwise. For example, reference to “a cell” includes a combination of two or more cells, and the like.

As used herein, “biological agent” refers to a natural compound. In some embodiments, the biological agent is combined with an ocular biomaterial to form an ocular biomaterial composition. In some embodiments, the biological agent provides structure to the ocular biomaterial composition. In some embodiments, the combination of the biological agent and ocular biomaterial is formed as a cell scaffold. By way of example, but not by way of limitation, biological agents include, but are not limited to, laminin and collagen.

As used herein, “cell delivery matrix” refers to a composition that includes an ocular biomaterial composition and cells. In some embodiments, the cell delivery matrix is a cell carrier that provides a matrix for cell survival, cell growth, cell differentiation, and/or cell integration. In some embodiments, the cell delivery matrix is delivered/implanted into the eye, e.g., by injection. By way of example, but not by way of limitation, an exemplary cell delivery matrix includes a retina-based biomaterial composition seeded with human retinal progenitor cells.

As used herein, “decellularized” refers to substantially removing cellular material from the cell or tissue source from which the ocular biomaterial matrix is derived, e.g., retina or interphotoreceptor matrix. In some embodiments, the decellularization process also removes other biological inhibitory components. By way of example, and not by limitation, in some embodiments, a decellularized ocular biomaterial is free of materials that would interfere with the survival, proliferation, differentiation, or integration of cells seeded in the ocular biomaterial matrix. Such interfering materials may include enzymes, hormones and other proteinaceous and nonproteinaceous molecules.

As used herein, “effective amount” or “therapeutically effective amount” refers to a quantity sufficient to achieve a desired therapeutic and/or prophylactic effect, e.g., an amount which eliminates, reduces, or ameliorates symptoms associated with degenerative eye diseases or orders. Symptoms of degenerative eye diseases or orders include, but are not limited to decrease night vision, reduced peripheral vision, narrow field of vision, tunnel vision, night blindness, reduced vision, blindness, blurred vision, spots or dark strings in field of vision, loss of color perception, distorted vision, and loss of central vision.

As used herein, “biological inhibitory components” refers to compounds that inhibit cell growth, cell survival, cell differentiation, cell migration, or nerve or retina regeneration. By way of example, but not by limitation, in some embodiments, biological inhibitory components include, but are not limited to, chondroitin sulfate, proteoglycans, and leukocyte antigens.

As used herein, “ocular biomaterial” refers to a biomaterial isolated from or derived from the eye. In some embodiments, ocular biomaterial is isolated from an eye (e.g., dissected from an eye). Exemplary biomaterial include, but is not limited, to retinal tissue, interphotoreceptor matrix, vitreous humor, retinal progenitor cells, cornea cells, retinal pigment epithelium cells, choroid cells, and macular cells. In some embodiments, ocular biomaterial such as cells can be derived from the eye, and then cultured in vitro (e.g., cultured cells derived from eye). The cell progeny of the original isolates are considered “ocular biomaterial.” After isolation or dissection, ocular biomaterial may treated by any number of methods, depending on the intended use. By way of example, but not by way of limitation, in some embodiments, ocular biomaterial is isolated, dried and/or ground and/or lyophilized and/or suspended in a liquid medium. In some embodiments, ocular biomaterial is combined with other components to form an ocular biomaterial composition. In some embodiments, the ocular biomaterial composition may be a liquid, gel, film, semi-solid or solid structure, scaffold, or may be formed onto an implant, with shape and consistency appropriate for intended use. In some embodiments, the ocular compositions may be seeded with cells to form a matrix. In some embodiments, the matrix is in the form of a liquid, gel, film, semi-solid or solid structure, or may be formed onto an implant, with shape and consistency appropriate for intended use.

As used herein, “prevention” or “preventing” of a disorder or condition refers to a compound that, in a statistical sample, reduces the occurrence of the disorder or condition in the treated sample relative to an untreated control sample, or delays or prevents the onset of one or more symptoms of the disorder or condition relative to the untreated control sample. As used herein, preventing degenerative eye diseases or disorders includes preventing the initiation of degenerative eye diseases or disorders, delaying the initiation of degenerative eye diseases or disorders, delaying the progression or advancement of degenerative eye diseases or disorders. As used herein, prevention of degenerative eye diseases or disorders also includes preventing an occurrence or re-occurrence of degenerative eye diseases or disorders.

As used herein, the terms “treating” or “treatment” or “alleviation” refers to therapeutic treatment, wherein the object is to slow down (lessen), reverse, or treat degenerative eye diseases or disorders and their associated symptoms. A subject is successfully “treated” for degenerative eye diseases or disorders if, after receiving a therapeutic amount of the cell delivery matrix according to the methods described herein, the subject shows observable and/or measurable reduction in or absence of one or more signs and/or symptoms of degenerative eye diseases or disorders, such as, e.g., night vision, reduced peripheral vision, narrow field of vision, tunnel vision, night blindness, reduced vision, blindness, blurred vision, spots or dark strings in field of vision, loss of color perception, distorted vision, and loss of central vision. It is also to be appreciated that the various modes of treatment or prevention of medical conditions as described are intended to mean “substantial,” which includes total but also less than total treatment or prevention, and wherein some biologically or medically relevant result is achieved.

### Ocular Biomaterial Compositions

General

Unlike fish and amphibians, which can regenerate retina, adult mammalian retina does not possess the ability to self-repair following injury or disease. Retinal degeneration and associated photoreceptor loss is the leading cause of blindness in the industrialized world. It is estimated that 25 to 30 million people worldwide suffer from age-related macular degeneration (AMD). Vugler et al., Mechanisms of development, 124(11-12): 807-829 (2007). Current treatment strategies, which focus mainly on slowing the degeneration process, include gene therapy, anti-angiogenic therapy, and growth factor treatment.

While it is not currently clear what specific biological factors enable retinal regeneration in certain amphibian and fish species, micro-environmental extracellular matrix (ECM) cues are likely play a role.

Cell transplantation is a more recent approach for retinal repair. Reports indicate that cells implanted into the eye can survive, differentiate into photoreceptors, integrate into retinal tissue, and improve visual function. See, e.g., Klassen et al., Investigative Ophthalmology & Visual Science, 45(11): 4167-4173 (2004) and Vugler et al., Mechanisms of development, 124(11-12): 807-829 (2007). The retina is an accessible compartment of the central nervous system, and is considered “immune privileged,” making it a candidate for cell-based therapies. One approach for retinal repair is the transplantation of retinal progenitor cells (RPC) into the subretinal space.

Cell delivery and survival is a challenge in retinal cell transplantation. Bolus injection of a cell suspension is the conventional method for subretinal cell transplantation. See, e.g., Tomita et al., Stem cells, 23(10):1579-1588 (2005). However, subretinal cell injection results in high (>99%) cell loss due to death and efflux during and post-transplantation. See, e.g., Redenti et al., Journal of Ocular Biology, Diseases, and Informatics, 1:19-29 (2008). Many of the cells that survived remain in the subretinal space rather than integrate within the retinal tissue in a manner appropriate for enhancing visual function. For example, injection of tens of thousands of cells resulted in only hundreds of cells integrating into retinal tissue. MacLaren et al., Nature, 444(7116): 203-207 (2006).

In general, the present technology relates to compositions and methods that are useful for enhanced cell survival, cell proliferation, cell differentiation, and cell integration in retinal cell transplantation. In some embodiments, the present technology provides an ocular biomaterial composition useful for the delivery of cells to the eye.

Retina-Based Biomaterial Compositions

The retina is a light-sensitive multi-layered tissue, which lines the inner surface of the eye. The optics of the eye create an image of the visual world on the retina.

In some embodiments, the ocular biomaterial comprises a retina-based biomaterial. In some embodiments, the retina-based biomaterial includes isolated retina.

The retina can be isolated by any number of methods commonly used in the art. By way of example, but not by way of limitation, in some embodiments, the retina is isolated from at least one enucleated eye that is hemisected with scissors to remove the lens and anterior structures before separating the retina by peeling from the retinal pigment epithelium.

In some embodiments, the retina is from a mammal, an amphibian, or a fish. In some embodiments, the retina is from a species in which retinal regeneration occurs. In some embodiments, the retina is isolated from a species in which retinal regeneration either during a quiescence state or a regenerative state.

In some embodiments, the retina is selected based on developmental age of the mammal, amphibian, or fish. In some embodiments, the developmental age is an early developmental age.

In some embodiments, the retina-based biomaterial composition includes decellularized, isolated retina. In some embodiments, the isolated retina is decellularized using nonionic detergents, amphoteric detergents, anionic detergents, cationic detergent, or a combination thereof. In some embodiments, the isolated retina is decellularized using 1% sodium dodecyl sulfate (SDS).

Nonionic detergents include, but are not limited to, Tween 20, Tween 80 and Triton X-100. Amphoteric detergents include, but are not limited to, Empigen BB, sulfobetaine-10, sulfobetaine-16, and 3-[(3-cholamidopropyl)dimethylammonio-1-propanesulfonate (CHAPS). Anionic detergents include, but are not limited to, Triton X-200, dodecylbenzene sulfonate, sodium caprylate, and sodium deoxycholate. Cationic detergents include, but are not limited to, hexadecyltrimethylammonium bromide (CTAB).

The isolated retina can be decellularized by any number of methods commonly used in the art. By way of example, but not by way of limitation, in some embodiments, the method for decellularizing isolated retina includes incubating the isolated retina in a solution containing the detergent for 24 hours at room temperature on a vertical rotator at 10 rpm. In some embodiments, solution is the detergent in a 50 mM sodium buffer at pH 7.2-7.6. In some embodiments, the detergent is removed after the incubation by dialysis against de-ionized water for 48 hours.

In some embodiments, chondroitin sulfate proteoglycan is removed from decellularized, isolated retina. By way of example, but not by limitation, in some embodiments, chondroitin sulfate proteoglycan is removed from decellularized, isolated retina by incubating the decellularized, isolated retina in PBS containing 2 U/ml chondroitinase ABC for 16 hours at 37° C.

In some embodiments, the decellularized, isolated retina is lyophilized and pepsin digested. Lyophilization can be performed by any method commonly used in the art. By way of example, but not by way of limitation, in some embodiments, the decellularized, isolated retina is lyophilized by freeze drying the decellularized, isolated retina (e.g., freeze drying with a Flexi-Dry MP, Kinetics Thermal Systems, Stone Ridge, N.Y.) for 48-72 hours until the retina is completely dry, freezing the dried, decellularized, isolated retina in liquid nitrogen, grinding the frozen retina (e.g., with a mortar and pestle) to produce a lyophilized powder.

Pepsin digestion can be performed by any method commonly used in the art. By way of example, but not by way of limitation, in some embodiments, the lyophilized powder is pepsin digested by adding between about 1 mg to 50 mg, or about 5 mg to 45 mg, or about 10 mg to 40 mg, or about 15 mg to 35 mg, or about 20 mg to 30 mg of the lyophilized retina powder with 1 mg of pepsin in 0.01N HCL solution for 48 hours at room temperature under constant stirring and diluting the digestion solution with 10×PBS and 0.1N NaOH. In some embodiments, the digestion solution is diluted to obtain a final retina ECM concentration of between about 1-30 mg/ml, or about 5-25 mg/ml, or about 10-20 mg/ml, or about 14-16 mg/ml. In some embodiments, the pepsin digested, decellularized, isolated retina contains similar collagen, sulfated-GAG, and hyaluronic acid levels as untreated retina.

In some embodiments, the retina-based biomaterial composition, e.g., a decellularized, isolated retina that has been lyophilized and pepsin digested, is acellular so as to avoid the adverse host immune response while retaining bioactive molecules. In some embodiments, the bioactive molecules include growth factors. In some embodiments, growth factors include, but are not limited to, basic fibroblastic growth factor (b-FGF), epidermal growth factor (EGF) and nerve growth factor (NGF).

In some embodiments, the retina-based biomaterial composition, e.g., a decellularized, isolated retina that has been lyophilized and pepsin digested, is substantially free of one or more biologically inhibitory compounds. Biologically inhibitory compounds include, but are not limited to, chondroitin sulfate, proteoglycans, and leukocyte antigens

Interphotoreceptor Matrix-Based Biomaterial Compositions

The interphotoreceptor matrix (IPM) is located between photoreceptors and retinal pigment epithelium in the retina and is involved in functions of the visual cycle. The functions of the IPM include visual pigment chromophore exchange, retinal adhesion, metabolite trafficking, and growth factor presentation. The IPM has an intricate honeycomb-like structure and is highly heterogeneous in composition, as demonstrated by differential staining using probes including antibodies and lectins. The IPM contains both water soluble and water insoluble components, including proteins, glycoproteins, glycosaminoglycans and proteoglycans.

In some embodiments, the ocular biomaterial composition comprises an IPM-based biomaterial. In some embodiments, the IPM-based biomaterial includes isolated IPM.

The IPM can be isolated by any number of methods commonly used in the art. In some embodiments, the IPM is from isolated retina (see above for methods for isolating retina). By way of example, but not by limitation, in some embodiments, the IPM is isolated by immersing isolated retina in de-ionized water, wherein the IPM delaminates from the photoreceptor outer segments as a sheet. In some embodiments, the isolate retina is immersed in de-ionized water for about 10 minutes at 4° C. In some embodiments, the IPM is maintained in a hydrated state throughout the IPM isolation process. Typically, maintaining the hydrated state of the IPM preserves the extracellular matrix (ECM) ultrastructure, which, in some embodiments, impacts cellular attachment and infiltration of cells during implantation of a cell delivery matrix containing IPM-based biomaterial composition.

In some embodiments, the isolated IPM is from a mammal, an amphibian, or a fish. In some embodiments, the IPM is from a species in which retinal regeneration occurs. In some embodiments, the IPM is isolated from a species in which retinal regeneration either during a quiescence state or a regenerative state.

In some embodiments, the IPM is selected based on developmental age of the mammal, amphibian, or fish. In some embodiments, the developmental age is an early developmental age.

In some embodiments, the IPM-based biomaterial composition includes decellularized, isolated IPM. In some embodiments, the IPM is decellularized during the delamination of the IPM from the retina. Alternatively, or additionally, in some embodiments, the IPM is decellularized using nonionic detergents, amphoteric detergents, anionic detergents, cationic detergent, or a combination thereof.

Nonionic detergents include, but are not limited to, Tween 20, Tween 80 and Triton X-100. Amphoteric detergents include, but are not limited to, Empigen BB, sulfobetaine-10, sulfobetaine-16, and 3-[(3-cholamidopropyl)dimethylammonio-1-propanesulfonate (CHAPS). Anionic detergents include, but are not limited to, Triton X-200, dodecylbenzene sulfonate, sodium caprylate, and sodium deoxycholate. Cationic detergents include, but are not limited to, hexadecyltrimethylammonium bromide (CTAB).

The isolated IPM can be decellularized by any number of methods commonly used in the art. By way of example, but not by way of limitation, in some embodiments, the method for decellularizing isolated IPM includes incubating the isolated retina in a solution containing the detergent for 24 hours at room temperature on a vertical rotator at 10 rpm. In some embodiments, the solution comprises a detergent in a 50 mM sodium buffer at pH 7.2-7.6. In some embodiments, the detergent is removed after the incubation by dialysis against de-ionized water for 48 hours.

Chondroitin sulfate proteoglycan is known to inhibit nerve and retinal regeneration. In some embodiments, chondroitin sulfate proteoglycan is removed from decellularized, isolated IPM. By way of example, but not by limitation, in some embodiments, chondroitin sulfate proteoglycan is removed from decellularized, isolated IPM by incubating the decellularized, isolated IPM in PBS containing 2 U/ml chondroitinase ABC for between 2-16 hours at 37° C.

Combination Ocular Biomaterial Compositions

In some embodiments, an ocular biomaterial composition includes a combination of any of the above described embodiments of retina-based biomaterials and any of the above described embodiments of the IPM-base biomaterials.

In some embodiments, a combination biomaterial composition is produced, wherein isolated retina and isolated IPM are combined and processed together to form a biomaterial composition by any of the above disclose methods. By way of example, but not by way of limitation, isolated retina and isolated IPM are combined, decellularized, lyophilized, and pepsin degraded.

Additional Components

In some embodiments, the ocular biomaterial composition includes additional components. In some embodiments, the additional components improve or enhance cell growth, cell migration, cell integration, cell migration, cell survival, or implantation of the ocular biomaterial. By way of example, but not by way of limitation, additional components include, but are not limited to, laminin, an antibiotic, an antiviral, an antifungal, growth factors, cytokines/chemokines, hormones, cell signaling molecules, a gelling agent, a protease inhibitor, cell media, and bovine serum proteins.

Structures Formed from Ocular Biomaterial

The ocular biomaterial compositions described above (e.g., retina-based, IPM-based, or combinations thereof) can be formed into various structures. In some embodiments, the ocular biomaterial composition contains between about 1 mg to 50 mg, or about 5 mg to 45 mg, or about 10 mg to 40 mg, or about 15 mg to 35 mg, or about 20 mg to 30 mg, or about 24 mg to 26 mg by dry weight of isolated retina, IPM, or combination thereof.

By way of example, but not by way of limitation, the ocular biomaterial compositions described herein can be provided as a three-dimensional structure (e.g., a cell scaffold), gel-forming liquid, a planar layer, a thin film, a gel, or a jelly-like matrix, or a sponge. The manipulation, formation, sizing, and shaping of the ocular biomaterial matrix depends on their ultimate mechanical and biological function. In some embodiments the ocular biomaterial compositions are provided to function as an extracellular matrix (ECM) that support the survival, proliferation, differentiation, and integration of cells, either in vitro or for transplant, e.g., in vivo use. Additionally or alternatively, ocular biomaterial compositions are provided to function on the surface of an ocular implant. Accordingly, the ocular biomaterial compositions can be formed into any number of structures or dimensions, based on intended use.

In some embodiments, an ocular biomaterial composition is combined with additional agents to form a cell scaffold.

In another embodiment, the ocular biomaterial composition is applied (e.g., as a coating) onto a substrate (e.g., an ocular implant) to form a cell scaffold. In some embodiments, the ocular biomaterial composition is chemically linked onto polymer backing material. In some embodiments, the ocular biomaterial composition is combined with additional agents before application to a substrate to form a cell scaffold. Substrates include, but are not limited to, a membrane (e.g., a polymer membrane or biological membrane), tissue culture inserts, ocular implants (e.g., retinal implant), and cell culture plates.

In some embodiments, the additional agents are biological agents. In some embodiments the biological agents provide structure to the ocular biomaterial compositions. By way of example, but not by way of limitation, collagen can be added to impart temperature sensitive gel formation. Examples of biological agents include, but are not limited to, collagen, fibronectin, and transglutaminase.

In some embodiments, the additional agents are synthetic agents. In some embodiments the synthetic agents provide structure to the ocular biomaterial compositions. By way of example, but not by way of limitation, polymers could be added to provide a polymer scaffold. In some embodiments, the polymers are neutral. Alternatively, or additionally, in some embodiments, the polymers are biodegradable. Examples of synthetic agents include, but are not limited to, polymers (e.g., poly(lactic-co-glycolic acid) (PLGA)), polyglycerol sebacate (PGS), poly(L-lactic acid) (PLLA), poly(methylmethacrylate) (PMMA), and polycaprolactone (PCL).

Cell scaffolds are used in the art for various applications, and their manipulation, formation, sizing, and shaping all depend on their ultimate mechanical and biological function. Thus, cells scaffolds of the present technology can have any number of structures or dimensions, based on intended use.

Exemplary methods for forming scaffolds comprising ocular biomaterial compositions are described below. These examples should in no way be construed as limiting the scope of the present technology.

Films:

In some embodiments, retina-based, IPM-based or a combination of retina and IPM-based biomaterial compositions are formed into films. By way of example, but not by way of limitation, in some embodiments, isolated retina or IPM is decellularized using 1% sodium dodecyl sulfate (SDS) and purified using dialysis for 48 hours against 5 changes of de-ionized water in 12 Kda MWCO cassettes. In some embodiments, the treated retinal or IPM biomaterial composition is in liquid form and is casted onto a mold, or flat surface (e.g., tissue culture plate) at room temperature. In some embodiments, the solution is dried to form a film. In some embodiments, the dried film composition is then peeled from the mold or flat surface. In some embodiments, the film is then sterilized using 70% ethanol and/or UV light.

Gels:

In some embodiments, retina-based, IPM-based or a combination of retina and IPM-based biomaterial compositions are formed into gels. By way of example, but not by way of limitation, in some embodiments, isolated retina and/or IPM is decellularized, lyophilized and pepsin digested. By way of example, but not by limitation, in some embodiments, a method for producing a retina-based, IPM-based or combination gel includes digesting between about 1 to 40 mg, or about 5 to 35 mg, or about 10 to 30 mg, or about 15 to 25 mg, or about 18-22 mg of lyophilized, decellularized, isolated ocular biomaterial with about 1 mg of pepsin in 0.01N HCl for 48 hours at room temperature under constant stirring, diluting the digestion solution with 10×PBS and 0.1N NaOH at 4° C., and gelling the diluted solution at 37° C. for 30 minutes.

Scaffolds:

In some embodiments, retina-based, IPM-based or a combination of retina and IPM-based biomaterial compositions are formed into scaffolds. By way of example, but not by limitation, in some embodiments, an IPM-based, retina based or combination biomaterial cell scaffold is produced by suspending 100 μl of isolated IPM and/or decellularized, isolated retina in de-ionized water or sterile PBS and centrifuging the solution at 2500 rpm for 20 minutes at 25° C. onto tissue culture inserts (e.g., polyethylene terephthalate (PET) membranes) or culture plates. In some embodiments, the suspension containing the isolated IPM and/or decellularized, isolated retina is pre-centrifuged at 5000 rpm for 20 minutes, at 25° C. before centrifugation onto tissue culture inserts or culture plates.

### Ocular Biomaterial Compositions Comprising Cells

In some embodiments, the above described ocular biomaterial compositions are seeded with cells to form a cell delivery matrix. In some embodiments, the ocular biomaterial compositions of the matrix enhance cell growth, cell survival, cell differentiation, and/or cell integration of the seeded cells into the native tissue (e.g., upon transplantation), as compared to cells not seeded in the compositions.

In some embodiments, the cell delivery matrix is used as a carrier to deliver cells, e.g., via implantation, to the eye. In some embodiments, the cell delivery matrix enhances cell growth, cell survival, cell differentiation, and/or cell integration of seeded cells into the eye, as compared to cells delivered by a carrier not including the ocular biomaterial compositions of the present disclosure.

In some embodiments, the cells seeded in the ocular biomaterial compositions include, but are not limited to, retinal cells, retinal progenitor cells, retinal stem cells, embryonic stem cells (ESCs), retinal pigment epithelium, glial cells, and stem cells. In some embodiments, the cells are mammalian. In some embodiments, the cells are human. In some embodiments, the cells are ocular cells. In some embodiments, the cells are human retinal progenitor cells (hRPCs).

By way of example, but not by limitation, an exemplary method for seeding cells to form a cell delivery matrix (e.g., a retina-based biomaterial composition, a IPM-based biomaterial composition, or a combination thereof) includes, triple rinsing a prepared ocular biomaterial film in PBS, pre-treating the ocular biomaterial film with cell culture medium in cell culture plates before cell seeding and seeding the cells, e.g., hRPCs onto the film.

Additionally or alternatively, in some embodiments, seeding cells into an ocular biomaterial composition includes sterilizing a solution containing decellularized, isolated retina, decellularized, isolated IPM, or a combination thereof, combining the sterilized solution with a cell suspension, and incubating the solution/cell suspension for gelation. In some embodiments, the combing of the sterilized solution with a cell suspension is performed at 4° C. In some embodiments, the solution/cell suspension is pipetted into plates for gelation. In some embodiments, the gelation is performed at 37° C. for 30 minutes.

In some embodiments, the cell delivery matrix is implanted into the eye. Implantation of the cell delivery matrix can be performed by methods known in the art. By way of example, but not by limitation, in some embodiments, the cell delivery matrix is implanted by injection into the eye, e.g., by a syringe. Additionally or alternatively, in some embodiments, the cell delivery matrix is implanted as a film. In some embodiments, the cell delivery matrix is implanted into the subretinal space of the eye, directly injected into the retina, or laid onto the retina.

In some embodiments, the cell delivery matrix is applied (e.g., as a coating) onto a substrate (e.g., an ocular implant). Substrates include, but are not limited to, a membrane (e.g., a polymer membrane or biological membrane), tissue culture inserts, ocular implants (e.g., retinal implant), and cell culture plates.

### Prophylactic and Therapeutic Uses of Ocular Biomaterial Compositions

General

The ocular biomaterial compositions (e.g., ocular biomaterial matrices) described herein are useful to prevent or treat degenerative eye diseases or disorders. Degenerative eye diseases or disorders include, not are not limited to, macular degeneration (e.g., age-related macular degeneration (AMD)), retinitis pigmentosa, diabetic retinopathy, hypertensive retinopathy, radiation retinopathy, solar retinopathy, and retinopathy of prematurity. By way of example, but not by limitation, in some embodiments, the disclosure provides for both prophylactic and therapeutic methods of treating a subject having or at risk of (susceptible to) AMD. Accordingly, the present methods provide for the prevention and/or treatment of degenerative eye diseases or disorders in a subject by administering an effective amount of an ocular biomaterial matrix based cell delivery matrices to a subject in need thereof.

Therapeutic Methods

In some embodiments, the present technology includes methods for treating degenerative eye diseases or disorders, e.g., retinopathies and retinitis pigmentosa, for therapeutic purposes. In therapeutic applications, a cell delivery matrix is implanted in at least one eye of a subject suspected of having or already suffering from degenerative eye diseases or disorders in an amount sufficient to cure, or at least partially arrest, the symptoms of the disease, including its complications and intermediate pathological phenotypes in development of the disease. As such, in some embodiments, the present technology provides methods of treating an individual having or suspected of having degenerative eye diseases or disorders, e.g., retinopathies and retinitis pigmentosa.

Subjects suffering from degenerative eye diseases or disorders can be identified by any or a combination of diagnostic or prognostic assays known in the art. For example, typical symptoms of degenerative eye diseases or disorders include, but are not limited to, decrease night vision, reduced peripheral vision, narrow field of vision, tunnel vision, night blindness, reduced vision, blindness, blurred vision, spots or dark strings in field of vision, loss of color perception, distorted vision, and loss of central vision.

Prophylactic Methods

In some aspects, the invention provides a method for preventing or reducing the likelihood or severity of degenerative eye diseases or disorders, e.g., retinopathies and retinitis pigmentosa, in a subject having or suspected of having degenerative eye diseases or disorders. Subjects at risk for degenerative eye diseases or disorders can be identified by, e.g., decrease night vision, reduced peripheral vision, narrow field of vision, tunnel vision, night blindness, reduced vision, blindness, blurred vision, spots or dark strings in field of vision, loss of color perception, distorted vision, and loss of central vision. In prophylactic applications, a cell delivery matrix is implanted in at least one eye of a subject susceptible to, or otherwise at risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Determination of the Biological Effect of Implanted Ocular Biomaterial Compositions

In various embodiments, suitable in vitro or in vivo assays are performed to determine the effect of an ocular biomaterial composition, e.g., a cell delivery matrix, on degenerative eye diseases or disorders, e.g., retinopathies and retinitis pigmentosa. In various embodiments, in vitro assays can be performed with representative animal models, to determine if a cell delivery matrix exerts the desired effect in reducing one or more symptoms of degenerative eye diseases or disorders, e.g., decreased night vision, reduced peripheral vision, narrow field of vision, tunnel vision, night blindness, reduced vision, blindness, blurred vision, spots or dark strings in field of vision, loss of color perception, distorted vision, and loss of central vision, thereby preventing or treating degenerative eye diseases or disorders. Cell delivery matrices for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

### Kits

In some embodiments, the present technology provides for a kit that includes at least one ocular biomaterial composition. In some embodiments, the ocular biomaterial composition is a retina-based biomaterial composition, an IPM-based biomaterial composition, or a combination thereof.

In some embodiments, the kit also includes media, solutions, or reagents for combining the ocular biomaterial composition to cells to form a cell delivery matrix. Additionally or alternatively, in some embodiments, the kit also includes cells. Cells include, but are not limited to, retinal cells, retinal progenitor cells, retinal stem cells, embryonic stem cells (ESCs), retinal pigment epithelium, glial cells, and stem cells. In some embodiments, the kit includes instructions for combining the ocular biomaterial matrix and cells to form the cell delivery matrix.

Additionally or alternatively, in some embodiments, the kit includes a tool or tools for implanting or delivering cell delivery matrix. In some embodiments, the kit also includes directions for implanting or delivering the cell delivery matrix.

### EXAMPLES

The following examples are provided to more fully illustrate various implementations of the present technology. These examples should in no way be construed as limiting the scope of the present technology.

### Example 1

### Formation of Retina-Based Biomaterial Films

This example shows an exemplary method for making retina-based biomaterial films.

Isolation of Retina

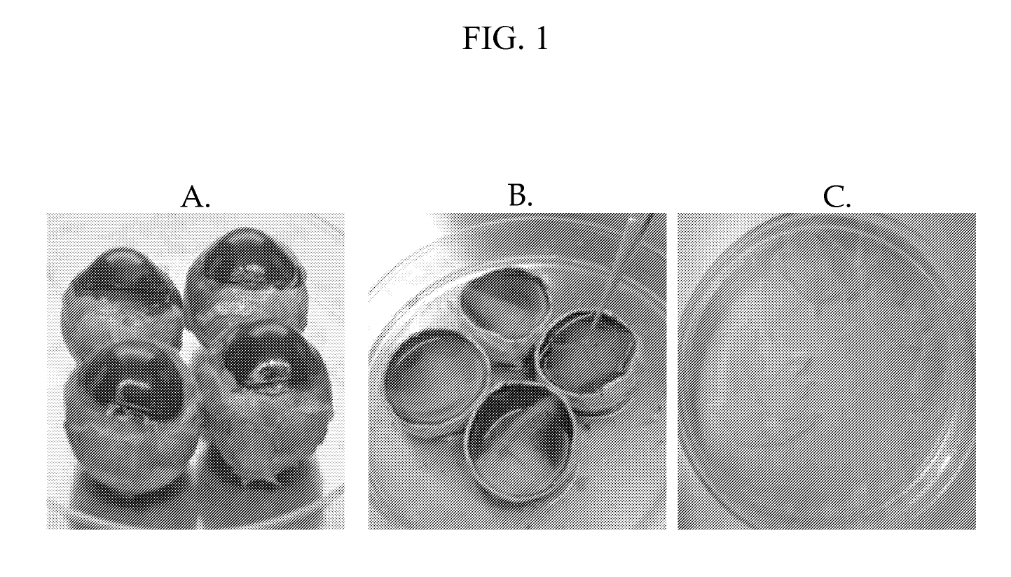

Enucleated bovine eye was hemisected with fine scissors to remove the lens, the cornea, and the vitreous humor. The hemisected eye-cup was flooded with phosphate buffer saline (PBS, Sigma-Aldrich) (FIG. 1) and the retina was peeled from the retinal pigment epithelium using a microspatula. The isolated retina was rinsed in sterile PBS for 7 minutes at 0° C. and then transferred to de-ionized (DI) water. (FIG. 1) The isolated retina was shaken on an orbital shaker (TECHNE Mini Orbital Shaker, TSSM1) at 75 rpm for 3 minutes at room temperature. The isolated retina was collected using transfer pipet and further processed for decellularization.

Decellularization of Retina

The isolated retinas from 8 eyes were transferred into a 250 ml conical flask containing 120 ml of 1% SDS solution at room temperature. The flask was shaken on an orbital shaker at 120 rpm for 3 hours at room temperature. The retina was then harvested using a transfer pipet. The harvested retina was then transferred into DI water and centrifuged at 25° C. at 10,000 rpm for 15 minutes. The retina solution was further purified using dialysis. The residual SDS was removed by loading the retina solution into a dialysis tubing (MWCO 12500, Sigma Aldrich) and dialyzing against de-ionized water with several changes (8-10 times over a span of 48 hours).

Lyophilization and Digestion of Retina

Decellularized retina was lyophilized and digested. The decellularized retina was lyophilized by freezing the retina solution from above in 50 ml falcon tubes in −80° C. freezer (Thermo Electron) overnight. The solution was lyophilized using a freeze dryer (Flexi-Dry MP) with the temperature maintained within the sample chamber between about −55 to −85° C. The decellularized retina samples were dried for 48-72 hours until complete removal of water/moisture. The lyophilized retina was snap-frozen in liquid nitrogen. Frozen pieces were grounded using a mortar and pestle to produce a lyophilized powder.

10 mg of the lyophilized decellularized retina was digested with 1 mg of pepsin (Sigma-Aldrich) in 0.01N HCl (Sigma-Aldrich) for 48 hours at room temperature under constant stirring. The digestion solution was diluted with 10×PBS and 0.1N NaOH to obtain a final retina concentration of 5-20 mg/ml.

Formation for Retina-Based Biomaterial Films

Retina-based biomaterial films were formed from decellularized, isolated retina. An isolated, decellularized retina solution was cast on a tissue culture plate surface at room temperature and dried overnight. The dried films were peeled from the molds and sterilized using 70% ethanol/UV light for an hour.

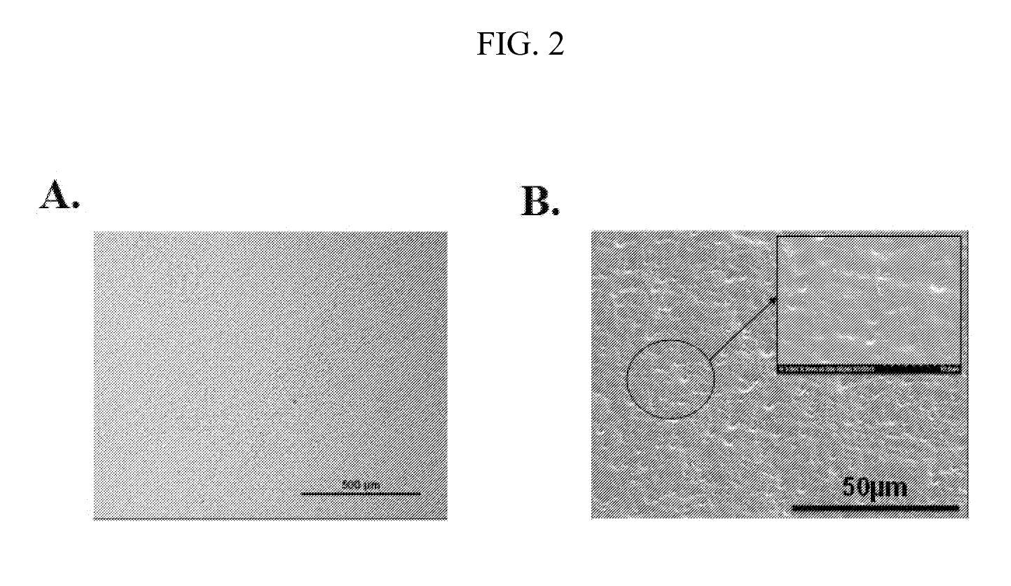

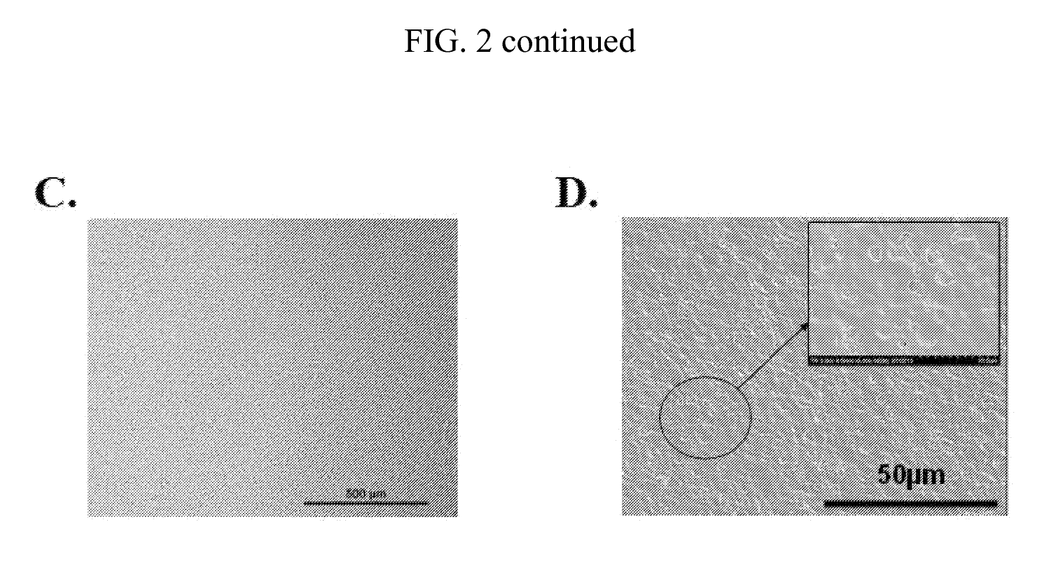

Retina-based biomaterial films were transparent and provided unhindered light transmission. The retina-based biomaterial films were easily removed and were easily handled. An ethanol treated retina-based biomaterial film was stable in PBS and possessed mechanical integrity. The thickness the retina-based biomaterial film was in the range of 10-20 μm. The surface topology of the retina-based biomaterial film (both untreated and ethanol treated) showed no visible differences under phase contrast microscope. FIG. 2A-D.

These results demonstrate that isolated retina can be processed into an ocular biomaterial matrix that can be formed into various structures.

### Example 2

### Biochemical Characterization of Retina-Based Biomaterial Composition

This example shows the biochemical characteristics of retina-based biomaterial composition.

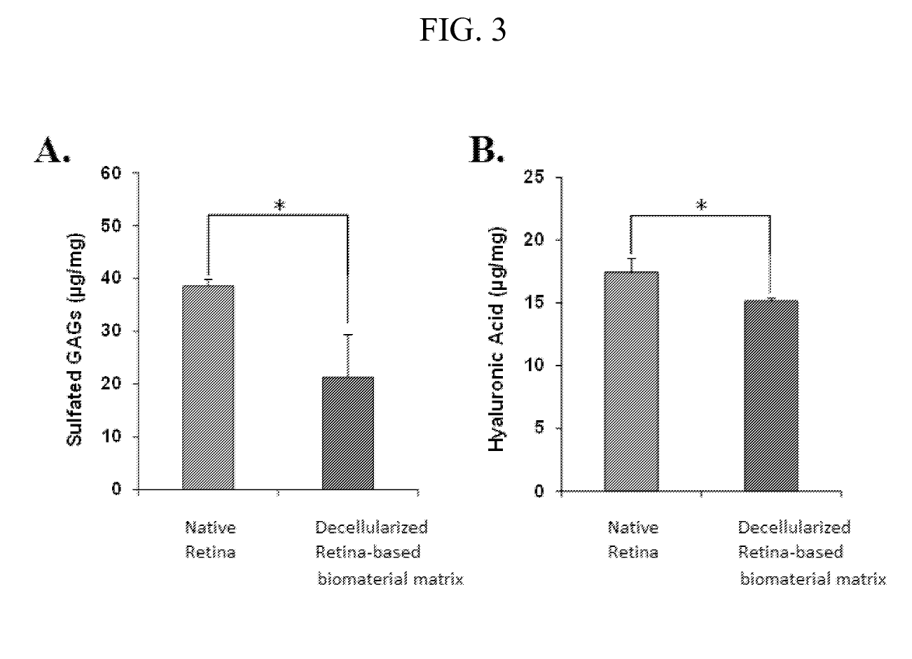

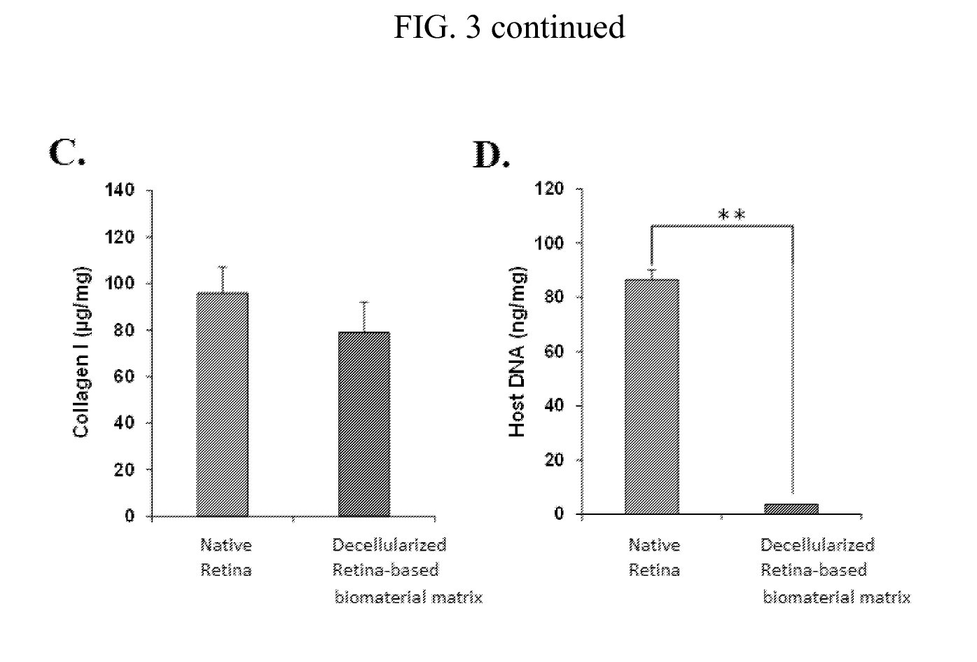

Sulfated glycosaminoglycan concentrations of pepsin digested and decellularized, isolated retina were quantified via spectrophotometry with 1,9-dimethylmethylene blue chloride (Sigma-Aldrich). The concentration of sulfated glycosaminoglycan was calculated based on a standard curve generated using chondroitin sulphate (Sigma-Aldrich). The absorbance at 492 nm was measured using a plate reader (Biotek, Powerwave XS, USA). The assay was performed in triplicates three times and the concentrations are represented as μg/mg dry weight. FIG. 3A.

The hyaluronic acid (HA) content of the pepsin digested and decellularized, isolated retina was measured using a Hyaluronan Enzyme-linked Immunosorbent assay kit (Echelon Biosciences, Salt Lake City, Utah) according to the manufacturer's protocol. The retina samples were mixed with HA detector and added to HA-ELISA plate for competitive binding. The enzyme-linked antibody and colorimetric detection was used to detect the HA detector bound to the plate. The concentration of HA in the sample was determined using a standard curve of known amounts of HA. The colorimetric assay at 405 nm was measured using a plate reader (Biotek, Powerwave XS, USA). FIG. 3B.

The total collagen-I was measured using a Hydroxyproline assay kit (Sigma Aldrich), by determining the hydroxyproline contents of the pepsin digested native and decellularized retina biomaterial according to the manufacturer's protocol. The pepsin digested samples were acid hydrolyzed and reacted with p-dimethylaminobenzaldehyde and chloramine-T. The hydroxyproline content in the samples was calculated based on a standard curve generated following the kit protocol. The absorbance at 560 nm are measured using a plate reader (Biotek, Powerwave XS, USA). The assay was performed in triplicates three times and the concentrations are represented as μg/mg dry weight. FIG. 3C.

The host DNA content was determined with Hoechst 33258 dye. Measurements of fluorescence intensity were used to assess DNA content of the pepsin digested native and decellularized, isolated retina according using fluorescence spectrophotometer (Biotek, Synergy HT Multi-mode microplate reader, USA; excitation wave-length: 360 nm, emission wave-length: 465 nm). The standard curve for DNA was generated using calf thymus DNA. FIG. 3D.

Preparation of Urea-Heparin Extract for Growth Factor Assays:

400 mg of powdered decellularized retina was suspended in 6 ml of heparin extraction buffer consisting of 2 M urea and 5 mg/ml heparin in 50 mM Tris with protease inhibitor (1 mM phenyl methyl sulphonyl fluoride (PMSF), 5 mM Benzamide, and 10 mM N-Ethylmaleimide (NEM)) at pH 7.4. The extraction mixture was rocked at 4° C. for 24 hours and then centrifuged at 12000×g for 30 minutes at 4° C. Supernatants were collected and 6 ml of freshly prepared urea-heparin extraction buffer was added to each pellet. Pellets with extraction buffer were again rocked at 4° C., centrifuged at 12000×g for 30 minutes at 4° C. and supernatants were collected. Supernatants from first and second extractions were collected and dialyzed against de-ionized water in a Slide-A-Lyzer Dialysis cassettes (Pierce, Rockford, Ill.). The concentration of total protein in each dialyzed extract was determined by the Bicinchoninic acid (BCA) protein assay (Pierce, Rockford, Ill.) following the manufacturer protocol.

Growth Factor Assays:

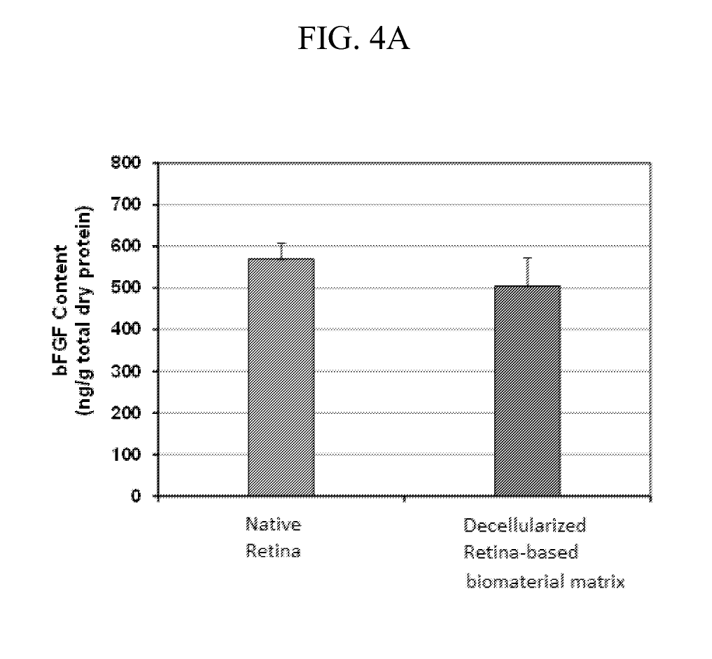

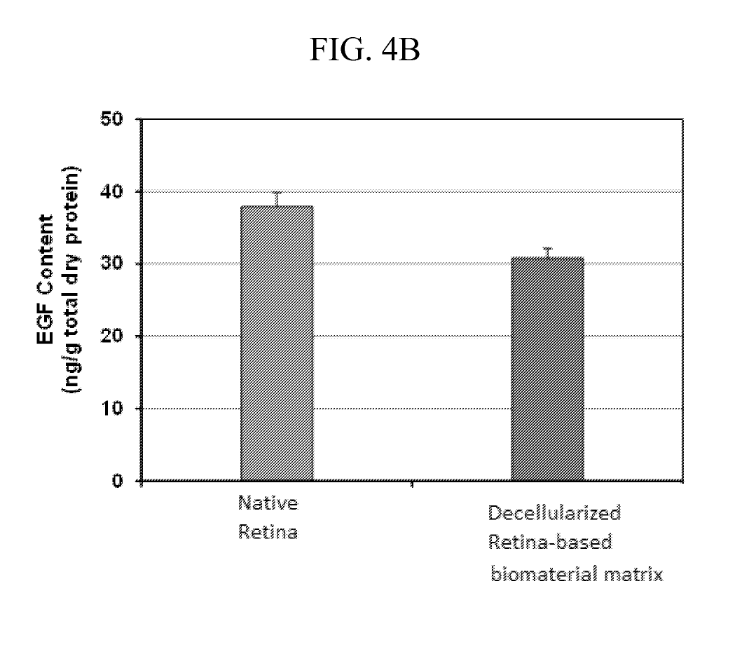

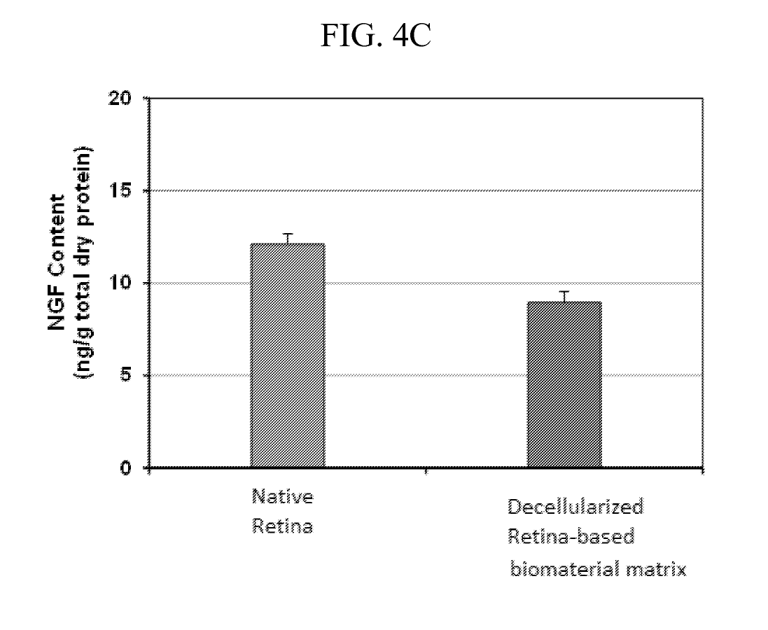

Concentrations of basic fibroblastic growth factor (bFGF) FIG. 4A, epidermal growth factor (EGF) FIG. 4B, and nerve growth factor (NGF) FIG. 4C in urea heparin extracts were determined using the human FGF-basic mini ELISA developmental kit (Peprotech, NJ, USA), human EGF mini ELISA development kit (Peprotech, NJ, USA) and the human β-NGF mini ELISA development kit (Peprotech, NJ, USA), respectively. Manufacturer instructions were followed for all three growth factor assays. Each assays for bFGF, EGF and β-NGF were done in triplicates. All the three assays measured the concentration of each growth factor and did not measure the growth factor activity.

Results

The pepsin digested decellularized retina-based biomaterial composition contained similar collagen (FIG. 3C), sulfated-GAG (FIG. 3A), and hyaluronic acid levels (FIG. 3B) as native retina, with the bulk of host DNA removed (FIG. 3D). The native retina composed of 95.9±11.3 μg of collagen I, 38.6±1.2 μg of s-GAGs, and 17.42±1.1 μg of hyaluronic acid per mg of initial dry weight of native retina. The decellularized retina-based biomaterial composition contained 79±13 μg of collagen, 21.3±8 μg of s-GAGs, and 15.14±0.22 μg of hyaluronic acid per mg of initial dry weight of decellularized-retina. The decellularization method was effective at decellularization of the retina. Decellularized-retina samples showed to contain <5 ng DNA per mg initial dry weight of decellularized-retina, which is significantly low as compared to native retina which contains ˜90 ng DNA per mg initial dry weight of native retina.

Analysis of decellularized retina-based biomaterial composition as compared to native retina showed high retention of growth factors after the decellularization process. The analysis shows that 569.5±38.07 ng of bFGF per mg initial dry weight of native retina was present as compared to 504.95±67.32 ng of bFGF after decellularization. The decellularized retina-based biomaterial composition (30.80±1.37 ng/g dry weight) showed a decrease of 20% of the EGF content as compared to native retina (37.89±1.93 ng/g dry weight). NGF was present at levels of 12.09±0.59 ng/g dry weight and 8.9±0.59 ng/g dry weight in native retina and decellularized retina-based biomaterial composition, respectively. See FIG. 4A (b-FGF), FIG. 4B (EGF), and FIG. 4C (NGF).

The results show that the retina-based biomaterial composition was acellular to avoid the adverse host immune response, while retaining the bioactive growth factors. The results also show that growth factors in the decellularized retina-based biomaterial composition were present in significant concentrations. The presence of growth factors indicate that the retina-based biomaterial composition supports cellular responses like cell attachment, survival, and proliferation. Accordingly, the ocular biomaterial composition of the present technology are useful for delivering cells for transplantation to eyes.

### Example 3

### Cell Growth and Survival in a Retina-Based Biomaterial Cell Delivery Matrix

This example shows that cells seeded in ocular biomaterial compositions have high survival rate and proliferate.

Human retinal progenitor cells (hRPCs) were isolated at a developmental stage optimal for differentiation and function upon integration with host tissue as described in Klassen et al., Investigative Ophthalmology & Visual Science, 45(11): 4167-4173 (2004). hRPCs cells and cell clusters were plated in flasks (Costar) coated with fibronectin (100 μg/ml for 30 minutes) in Ultraculture Media (Lonza), supplemented with 2 mM L-glutamine (Invitrogen), 20 ng/ml rh bFGF (Peprotech), 10 ng/ml rh EGF (Peprotech), and 1% antibiotic antimycotic solution x100 (Sigma-Aldrich). hRPCs was passaged at 80% confluence using tryspin-EDTA (Gibco-Invitrogen) with addition of benzonase (EMD Chemicals) and Defined Trypsin Inhibitor (Invitrogen). At each passage cell number and viability was estimated with Trypan blue (Sigma-Aldrich) staining using a hemacytometer with cells plated at a density of 10,000/cm². hRPCs at passage 7 were used to seed a retina-based biomaterial film.

Cell Seeding:

A prepared retina-based biomaterial film was triple rinsed in PBS and pre-treated with medium in culture plates for 1 hour at 37° C. prior to seeding with hRPCs. Cultured hRPCs were dissociated into single cell suspension and seeded on tissue culture plate surface (TCPS), retina-based biomaterial matrix film casted on TCPS, and fibronectin coated TCPS. Approximately, 100 μl/mm² (4×10³ cells/μl) of single-cell suspension were seeded and incubated at 37° C. at 5% CO₂. Fresh medium was added and changed every 2 days for 7 days.

Cell Morphology:

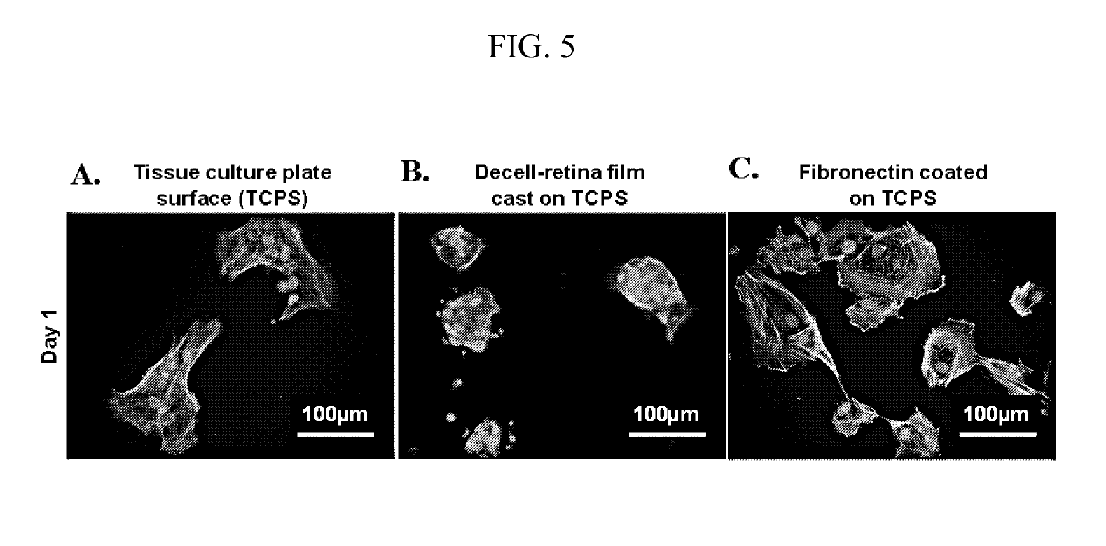

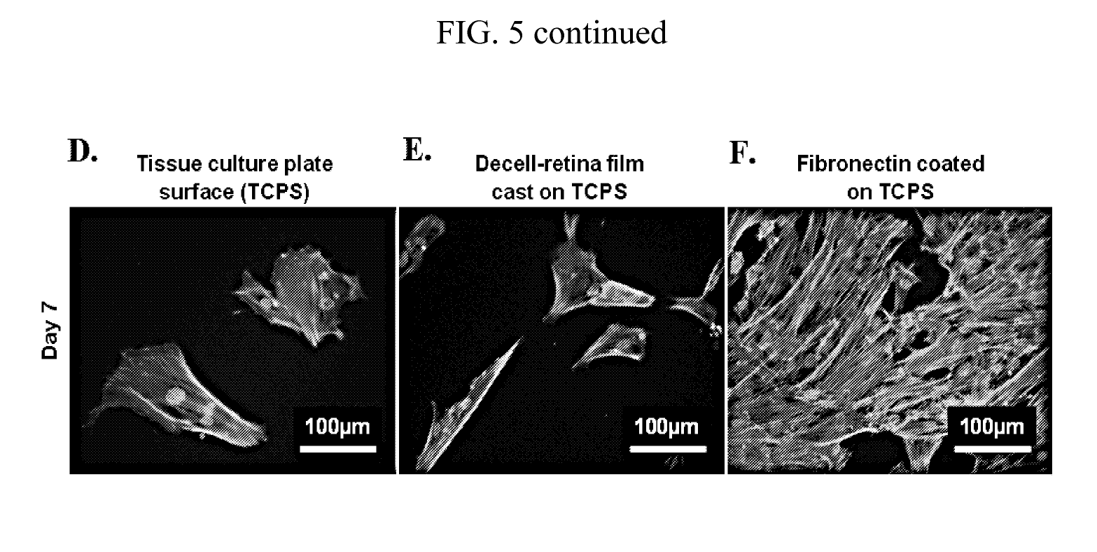

hRPCs cultured for 1 day and for 7 days (n=3) were fixed in 3.7% formaldehyde in PBS for 10 minutes at room temperature, then washed twice with PBS and permeabilized by 0.1% Triton X-100 (Sigma-Aldrich) in PBS for 5 minutes. After two washes with PBS, actin structures were labeled using phalloidin (Molecular Probes). Cells were incubated with 0.16 μM phalloidin in 1% BSA/PBS for 20 minutes at room temperature. Cells were counterstained with 5 μg/ml Hoechst 33258 (Molecular Probes) in PBS for another 20 minutes at room temperature. Cell cytoskeleton was observed using a fluorescence microscope (Olympus DP70), FIG. 5.

Gene Expression:

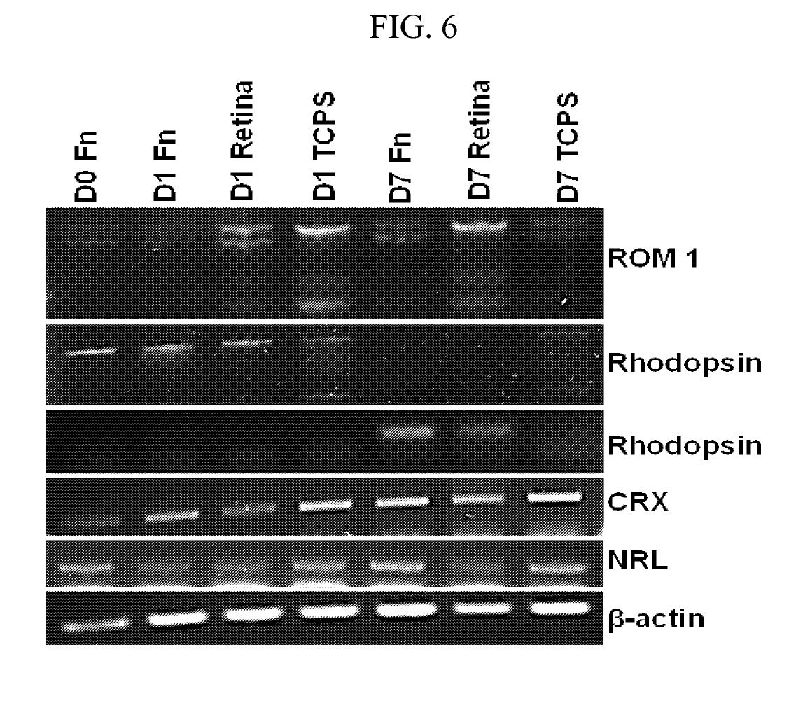

Total RNA was extracted from cell culture at day 1 and at day 7 using RNeasy Mini Kit (Qiagen) followed by column treatment with DNase I (Qiagen). Reverse transcription was performed with SuperScript III First-Strand Synthesis System (Invitrogen) and random primers (Sigma). The expression of the following genes were assayed: ROM1, Rhodopsin, CRX, NRL, and β-actin. The primer sequences (forward and reverse primers) used to identify the genes are shown Table 1. Real-time quantitative polymerase chain reaction (PCR) was performed with Mastercycler ep Realplex2 (Eppendorf North America) at 32 cycles with 35 ng of starting cDNA. The PCR products were identified using gel electrophoresis (2% agarose gel), FIG. 6.

TABLE 1
Primer sequences for the reverse transcriptase polymerase chain reaction.

| | | | Size |
|---|---|---|---|
| Gene | Forward Primer | Reverse Primer | (bp) |
| ROM 1 | TCCCTCCTGTCAGTTCCCTG | TAGAGCTGCATTCAGACTTGC | 115 |
| Rhodopsin | GTGCCCTTCTCCAATGCGA | TGAGGAAGTTGATGGGGAAGC | 202 |
| Crx | CCCCACTATTCTGTCAACGCC | GTCTGGGTACTGGGTCTTGG | 171 |
| NRL | GGCTCCACACCTTACAGCTC | GGCCCATCAACAGGGACTG | 212 |
| β3-actin | CATGTACGTTGCTATCCAGGC | CTCCTTAATGTCACGCACGAT | 250 |

Cell Viability:

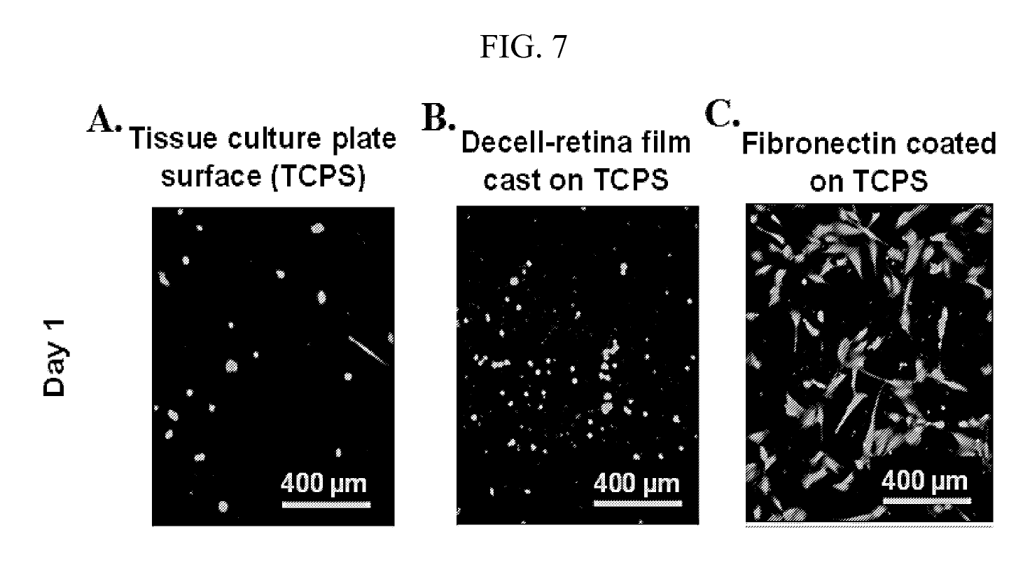

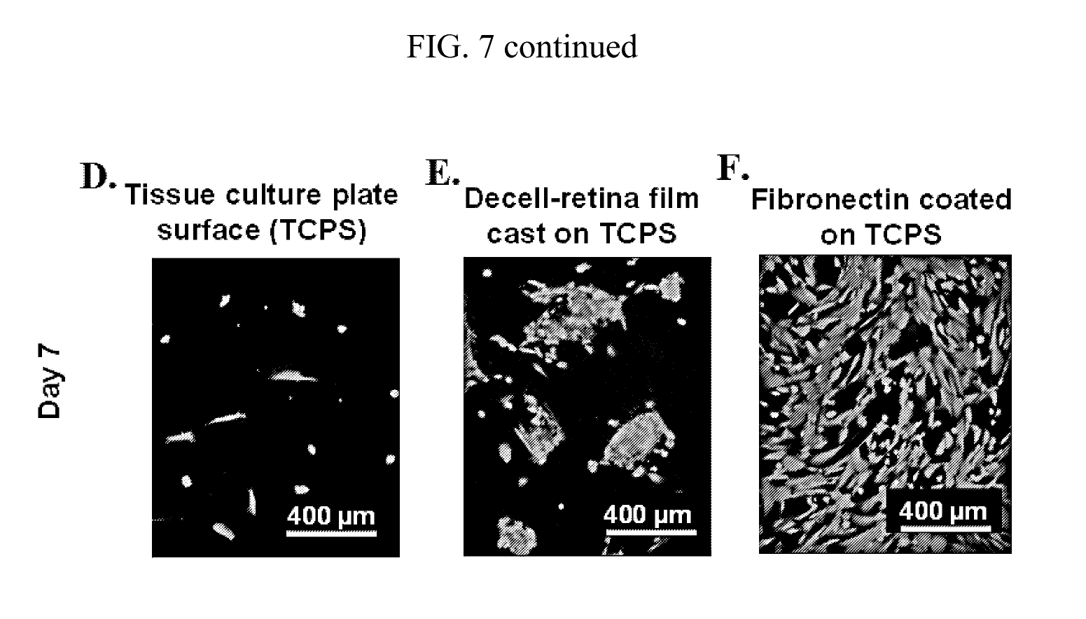

Cell viability studies were conducted using a Live/Dead Viability Kit (Invitrogen) using 2.0 μM calcein AM and 4.0 μM ethidium homodimer-1 (EthD-1). The assay dye was prepared by mixing 20 μl of EthD-1 and 5 μl of calcein AM in 10 ml sterile tissue culture grade D-PBS (Corning, USA). 200 μl of the combined Live/Dead assay reagent was added to the cells seeded on TCPS, retina-based biomaterial film casted on TCPS, and fibronectin coated TCPS at day 1 and at day 7 (n=3). The plates were incubated for 30 minutes at room temperature in dark. Following incubation and diluting with 100 μl of fresh D-PBS, the cells were observed under the fluorescent inverted microscope (Olympus, DP70). Calcein AM and EthD-1 was viewed simultaneously with a conventional fluorescein long pass filter. The fluorescence from the calcein was viewed with a standard fluorescein band pass filter (420-620 nm), while the EthD-1 was viewed with filters for Texas red dye (500-700 nm), FIG. 7A-F.

Cell Proliferation:

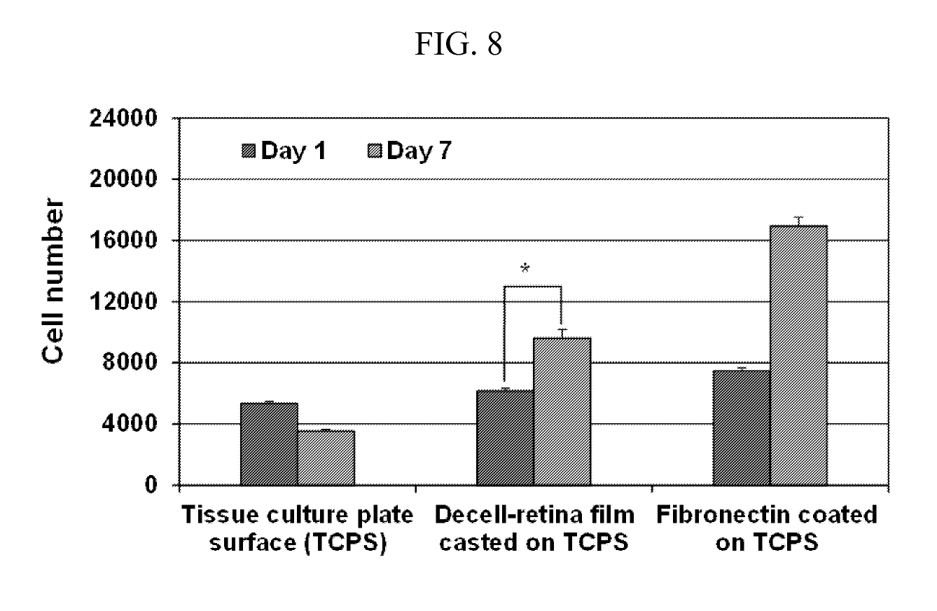

Cell numbers and DNA content of cultured cells at 1 day and 7 days (n=3) was quantified using the cyQUANT cell proliferation assay (Invitrogen), FIG. 8.

Statistical Analysis:

All the experimental data was expressed as mean±standard deviation. The significance was determined by the ANOVA test using MiniTab 15 software version. The value of p<0.05 was considered a significant difference.

Results

F-actin staining was performed to observe the morphology of the cells grown on TCPS, decellularized retina film cast on TCPS and fibronectin coated on TCPS after 1 and 7 days of culture (FIG. 5A-F). hRPCs cultured on the decellularized retina surfaces were found to grow as cell clusters after day 1 (FIG. 5B) and after day 7 (FIG. 5E), the cells were found to be elongated and spread out separately. hRPCs grown on the TCPS (FIG. 5A, 5D) and the fibronectin coated surface (FIG. 5C, 5F), were found to be spread out separately (actin filament development were more fibroblastic) and there was little difference in the cell morphology after day 1 and day 7 of culture. Morphological staining of the actin cytoskeleton and nucleus in the cells showed that the cells grew well on the decellularized retina surface.

Expression of Rhodopsin, retinal outer membrane-1 (ROM1), neural retina leucine zipper (NRL) and Cone-rod homeobox (CRX) genes on TCPS, decellularized retina film cast on TCPS, and fibronectin coated on TCPS after 1 and 7 days of culture was measure by real-time polymerase chain reaction (RT-PCR), FIG. 6. The expression level of ROM1 was higher on decellularized retina film, as compared to TCPS only and fibronectin coated on TCPS after day 7 of culture. The expression of rhodopsin did not differ much between the decellularized retina and fibronectin coated surfaces after day 7 of culture, while the TCPS did not show any expression for the rhodopsin gene. The expression level of NRL and CRX remains the same on all the surfaces after day 7 of culture. RT-PCR studies on the decellularized retina films showed expression of Rhodopsin, ROM1, NRL and CRX gene.

The cell viability results shows that the decellularized retina film surface is biocompatible and supports cell survival, FIG. 7A-F. The fluorescent imaging indicated a dominant population of live cells on the decellularized retina films (FIG. 7B, 7E). The results also indicate that the cell survival rate on the decellularized retina films were comparatively higher to that on TCPS (FIG. 7A, 7D). The fibronectin coated surface showed the maximum cell survival after day 7 of culture (FIG. 7C, 7F).

The number of cells were determined on the different surfaces (TCPS, decellularized retina film cast on TCPS, and fibronectin coated on TCPS for 1 and 7 days) after day 1 and day 7 of culture, FIG. 8. The hRPCs were found to proliferate (a 50% increase in cells) on the decellularized retina matrices from day 1 to day 7 of culture. The fibronectin coated surface showed the maximum cell proliferation between the three films. The cell proliferation rate on the decellularized retina film was significantly higher as compared to the cell proliferation rate on the TCPS.

These results show that the ocular biomaterial compositions of the present technology will support cell survival and proliferation. Accordingly, the ocular biomaterial compositions are useful as a cell delivery matrix for implantation of cells into the eye to prevent or treat degenerative eye diseases or disorders.

### Example 4

### Isolation of Interphotoreceptor (IPM) and Preparation of IPM-Based Biomaterial Matrix Cell Scaffold

This example shows an exemplary method for making a IPM-based biomaterial composition and an IPM-based biomaterial cell scaffold.

Isolation of IPM

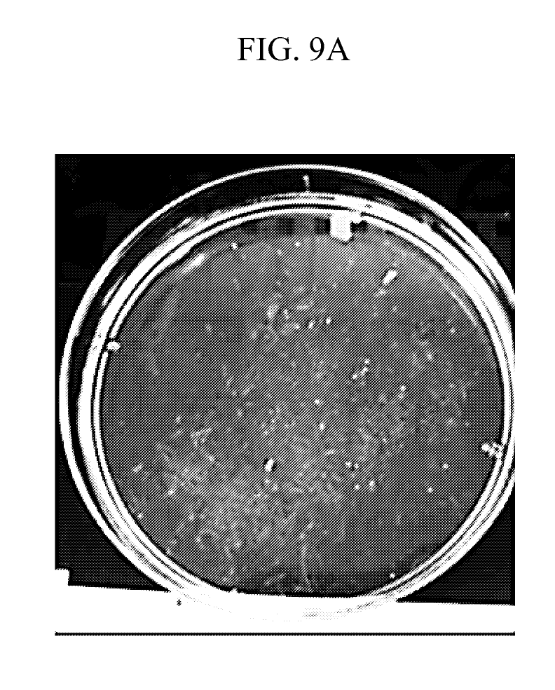

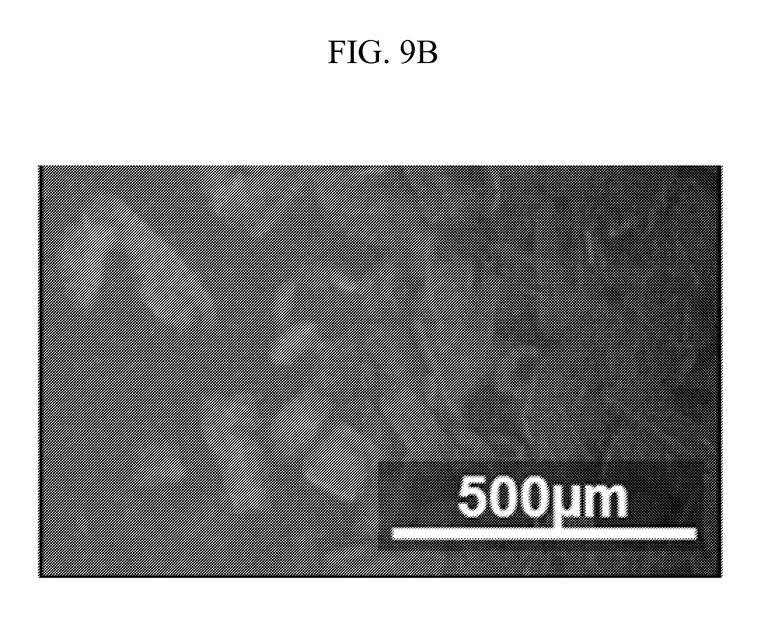

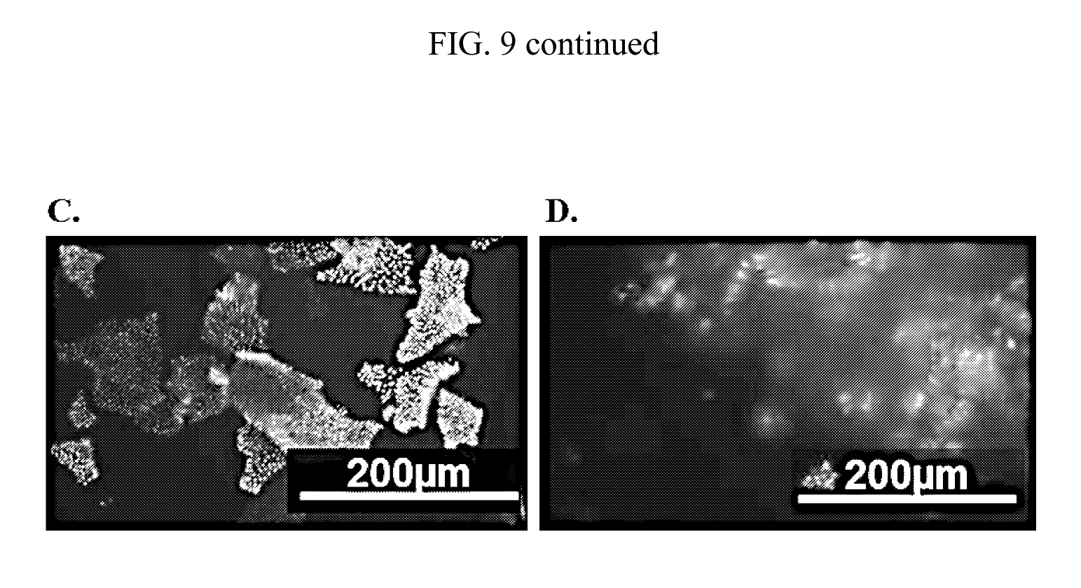

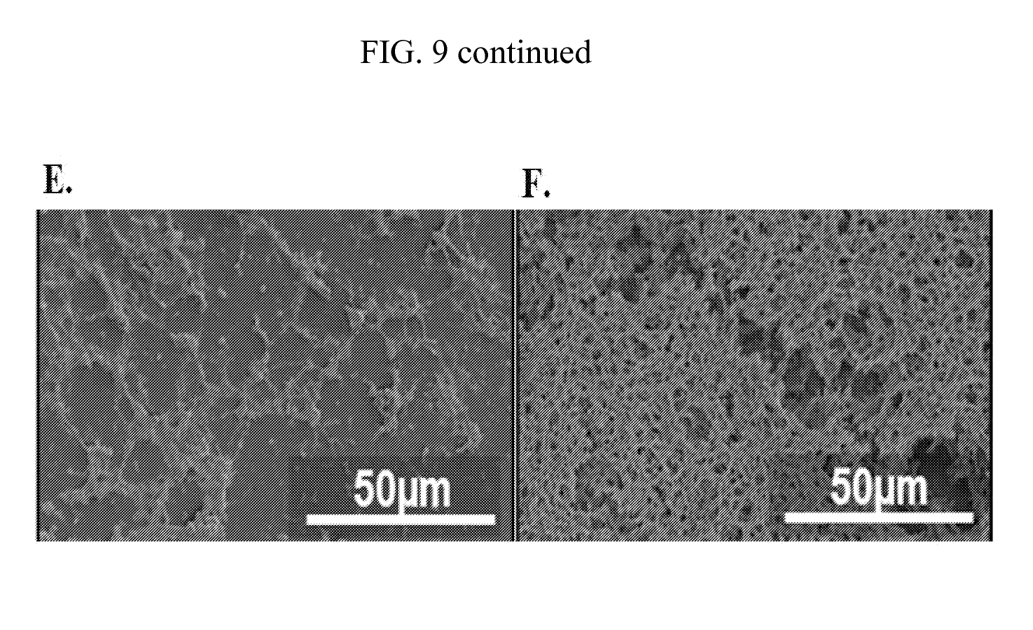

The IPM was isolated from adult bovine eyes (Research 87 Inc., Boston), collected from an abattoir within 3 hours after slaughter. The muscle surrounding the eye was removed and an incision was made 0.5 cm from the cornea circumferentially in order to separate the cornea and vitreous humor from the eye. The hemisected eye-cup was flooded with phosphate buffer saline (PBS, Sigma-Aldrich), and the retina was pulled away using a microspatula, while cutting the optic nerve to the retina. The whole neuroretina peeled from the retinal pigment epithelia was transferred into a sterile PBS solution. Using a transfer pipette, the isolated retina was transferred into de-ionized water (DI) in a large petri dish and shaken on an orbital shaker (TECHNE Mini Orbital Shaker, TSSM1) at 75 rpm for 3 minutes at room temperature. The IPM was readily isolated from the retina when placed in deionized water, in which the IPM floats off as structurally intact macro- to micro-scale sheets, FIG. 9A-B. The IPM was collected after removal of retina and any retina debris using a transfer pipet. The IPM that was collected after removal of retinas and any retina debris using a transfer pipet was referred to as “native IPM.” Concentrated IPM or “pre-centrifuged IPM” was prepared via centrifugation of the native IPM suspension at 4696×g for 20 minutes at 25° C.

Preparation of IPM-Based Biomaterial Cell Scaffold

IPM-based biomaterial cell scaffold was formed by centrifuging either native IPM or pre-centrifuged IPM suspended in sterile PBS onto tissue culture inserts (i.e., 0.4 μm polyethylene terephthalate (PET) membranes) (BD Falcon) in culture plates (Costar). The centrifugation was performed at 1174×g for 20 minutes at 25° C.

The IPM-based biomaterial cell scaffolds were treated with 2 U/ml of chondroitinase (Sigma-Aldrich) in PBS for 2 hours.

These results show that isolated IPM can be processed into an ocular biomaterial composition that can be formed into various structures.

### Example 5

### Biochemical Characterization of IPM-Based Biomaterial Matrix

In this example, the chemical characterization of “native IPM” from Example 4 was analyzed.

The total protein concentration of IPM-based biomaterial composition was measured using the BCA Protein Assay Kit (Thermo Scientific). The IPM-based biomaterial matrix was assayed per the manufacturer's protocol and the concentration was calculated based on a standard curve generated using albumin (Sigma-Aldrich), see Table 2. The absorbance at 562 nm was measured using a plate reader (Biotek, Powerwave XS, USA). The assay was performed in triplicate and the concentrations were represented as ng/mg dry weight.

Sulfated glycosaminoglycan concentrations of IPM-based biomaterial matrix was quantified via spectrophotometry with 1,9-dimethylmethylene blue chloride (Sigma-Aldrich). The IPM-based biomaterial matrix was assayed and the concentration was calculated based on a standard curve generated using chondroitin sulphate (Sigma-Aldrich). The absorbance at 492 nm was measured using a plate reader (Biotek, Powerwave XS, USA). The assay was performed in triplicate and the concentrations were represented as ng/mg dry weight.

The hyaluronic acid (HA) content of IPM-based biomaterial matrix was measured using a Hyaluronan Enzyme-linked Immunosorbent assay kit (Echelon Biosciences, Salt Lake City, Utah) according to the manufacturer's protocol. The IPM-based biomaterial matrix was mixed with HA detector and then added to HA-ELISA plate for competitive binding. Enzyme-linked antibody and colorimetric detection was used to detect the HA detector bound to the plate. The concentration of HA in the sample was determined using a standard curve of known amounts of HA. The colorimetric assay at 405 nm was measured using a plate reader (Biotek, Powerwave XS, USA).

The total collagen-I was measured using a Hydroxyproline assay kit (Sigma Aldrich), by determining the hydroxyproline contents of IPM according to the manufacturer's protocol. In brief, IPM was acid hydrolyzed and reacted with p-dimethylaminobenzaldehyde and chloramine-T. The hydroxyproline content in the samples was calculated based on a standard curve generated following the kit protocol. The absorbance at 560 nm was measured using a plate reader (Biotek, Powerwave XS, USA). The assay was performed in triplicates and the concentrations are represented as ng/mg dry weight.

Results

Biochemical characterization of the IPM-based biomaterial composition showed that there is a significant amount of sulfated GAGs and the non-sulfated GAGS, hyaluronic acid (HA) in the IPM-based biomaterial matrix. The IPM-based biomaterial composition was found to be mainly composed of sulfated GAGs (8±1 ng/mg), hyaluronic acid (60±10 ng/mg) and 0.149±0.001 μg/mg total protein content, Table 2. Collagen was not present at measureable levels in the IPM-based biomaterial composition, Table 2. The IPM-based biomaterial composition was thus mainly composed of glycoproteins and proteoglycans.

TABLE 2
Biochemical Composition of IPM

| Component | ng/mg of IPM |
|---|---|
| Sulfated GAG | 8.0 ± 1.0 |
| Hyaluronic Acid (HA) | 60.0 + 10.0 |
| Collagen | Not detectable |
| Protein | 149.0 + 1.0 |

HA provides the framework to which other components of the IPM are attached. The IPM does not possess high structural integrity due to its lack of collagen, which is unlike muscle, bone, cornea, and heart tissue, which contain reasonably high amounts of collagen fibrils.

These results show that IPM-based biomaterial composition are useful for the support of cell growth. Accordingly, the ocular biomaterial compositions disclosed herein are useful as a cell delivery matrix.

### Example 8

### Characterization IPM-Based Biomaterial Matrix Cell Scaffolds

Lectin Staining:

Lectin staining was performed on IPM-based biomaterial cells scaffolds described in Example 5, i.e., native IPM or pre-centrifuged IPM coated on a tissue culture insert. The IPM-based biomaterial cell scaffolds were treated with 0.1% Bovine Serum Albumin (Sigma) to prevent non-specific staining 50 μl of Fluorescein labeled Peanut Agglutinin (FPNA, Vector Laboratories) was added to each scaffold and the scaffolds were incubated for 30 minutes in the dark. The staining solution was removed and the scaffolds were rinsed with PBS. The surfaces were then examined under a fluorescence microscope (Olympus, DP70) with a standard fluorescein band pass filter (420-620 nm), FIG. 9C-D.

Scanning Electron Microscopy (SEM):

The surface morphologies of native IPM cells scaffolds and centrifuged IPM cells scaffolds were observed using an S-4800 Hitachi scanning electron microscope at an operating voltage of 3.0 KV. The native IPM cells scaffolds and centrifuged IPM cells scaffolds were dehydrated using sequential treatment with 35, 50, 70, 95 and 100% ethanol. After dehydrating with 100% ethanol, the samples were treated with hexamethyldisilazane (Sigma-Aldrich) and allowed to air dry. The dried samples were then attached to a specimen stub with a carbon adhesive tab, sputter coated with 15-nm gold-palladium, and examined at room temperature, FIG. 9E-F.

Results

Lectin staining of the isolated native IPM-based biomaterial composition shows that the structural features of the IPM as the fluorochrome conjugated lectin probes binds to the terminal carbohydrates in the composition. The lectin staining of the IPM-based biomaterial composition shows that IPM has an intricate honeycomb-like structure and is highly heterogeneous, FIG. 9C-D. The “honeycomb” structure is conducive to cellular growth. The pre-centrifuged IPM-based biomaterial composition lost some structural integrity during the centrifugation process, FIG. 9D.

SEM was utilized to characterize the surface of the IPM-based biomaterial cell scaffolds. SEM shows that the IPM-based biomaterial cell scaffolds consisted of a three-dimensional network of filaments of different sizes, possibly highlighting the assembly of different types of proteoglycans and glycoproteins, FIG. 9E-F. IPM-associated glycoconjugates participate in the adhesion between the retina and retinal pigment epithelium (RPE).

SEM also shows that pre-centrifugation of the IPM-based biomaterial composition before coating onto the tissue culture inserts was found to achieve a more homogeneous coating on the tissue culture insert, as compared to the patchy distribution of native IPM-based biomaterial composition coated tissue culture insert, FIG. 9E-F.

These results show that IPM-based biomaterial compositions have an internal structure that is useful for the support of cell growth. Accordingly, the ocular biomaterial composition disclosed herein are useful as a cell delivery matrix.

### Example 9

### Cell Growth and Survival in IPM-Based Biomaterial Cell Delivery Matrix

This examples shows that IPM-based biomaterial cell delivery matrices promote cell survival, cell growth, and differentiation.

Human retinal progenitor cells (hRPCs) were isolated from human fetal neural retina at 16 weeks of gestational age as described in Klassen et al., Investigative Ophthalmology & Visual Science, 45(11): 4167-4173 (2004). hRPCs cells and cell clusters were plated in flasks (Costar) coated with fibronectin (100 μg/ml) for 30 minutes in Ultraculture Media (Lonza), supplemented with 2 mM L-glutamine (Invitrogen), 20 ng/ml rh-bFGF (Peprotech), 10 ng/ml rh-EGF (Peprotech), and 1% antibiotic antimycotic solution x 100 (Sigma-Aldrich). hRPCs were passaged at 80% confluence using Tryspin-EDTA (Gibco-Invitrogen) with addition of benzonase (EMD Chemicals) and Defined Trypsin Inhibitor (Invitrogen). At each passage, cell number and viability was estimated with Trypan blue (Sigma-Aldrich) staining using a hemacytometer. For all experiments described below, hRPCs at passage 7 were assayed, except cells in immunocytochemical studies, which were used at passage 10.

Cell Seeding:

IPM-based biomaterial cell scaffolds were formed as described in Example 5 with native IPM, native IPM treated with chondroitinase, pre-centrifuged IPM and pre-centrifuged IPM treated with chondroitinase. The IPM-based biomaterial cell scaffolds were triple rinsed in PBS and pre-treated with medium in culture plates for 1 hour at 37° C. prior to seeding. Cultured hRPCs were dissociated into single cell suspension and seeded on to the cell scaffolds. Approximately 100 μl/mm² (4×10³ cells/μl) of single cell suspension was seeded and incubated at 37° C. at 5% CO₂. Fresh medium was added and changed every 2 days for 7 days.

Cell Viability:

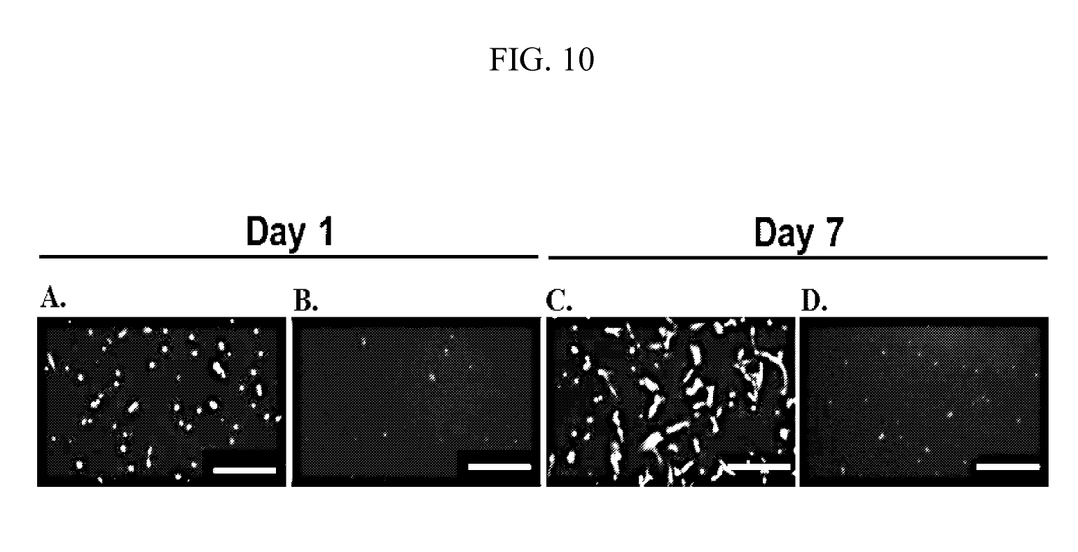

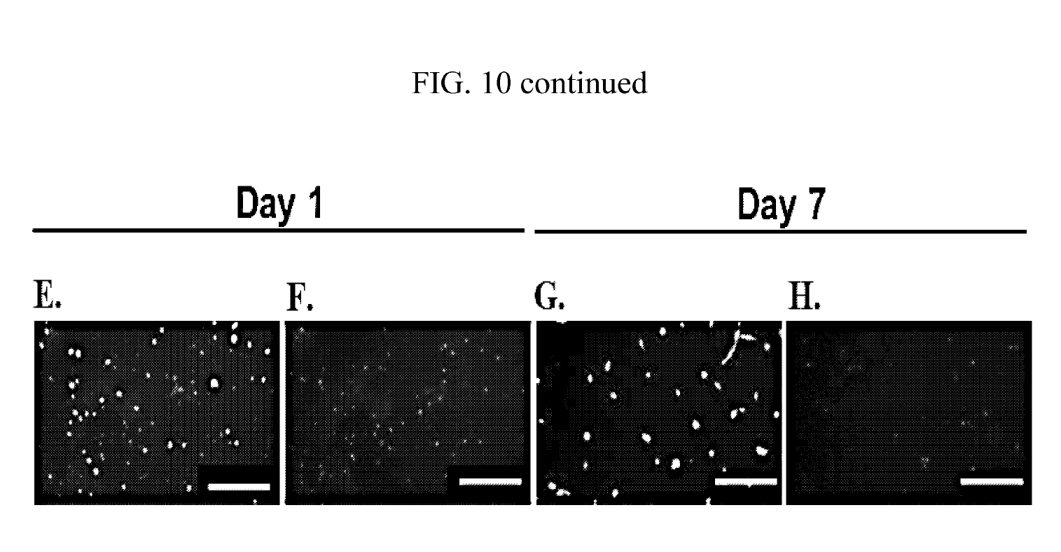

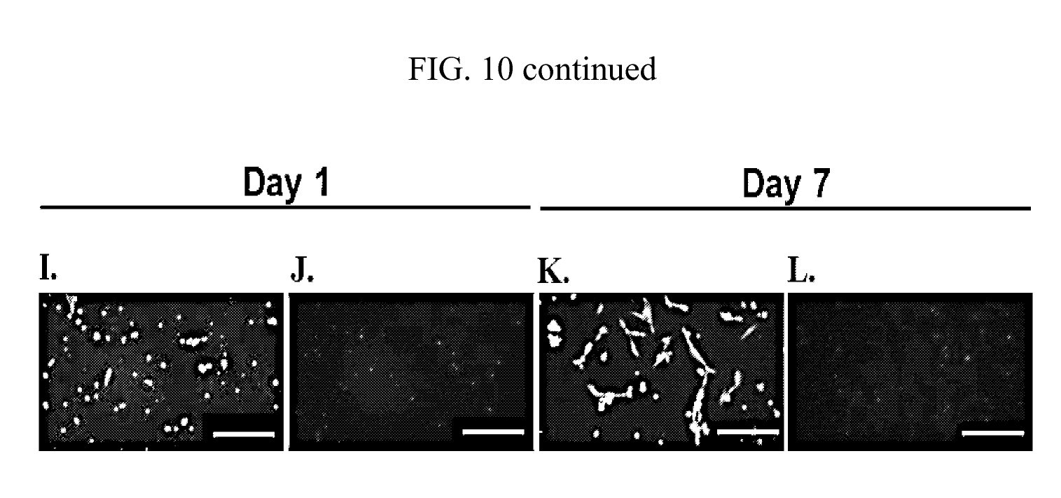

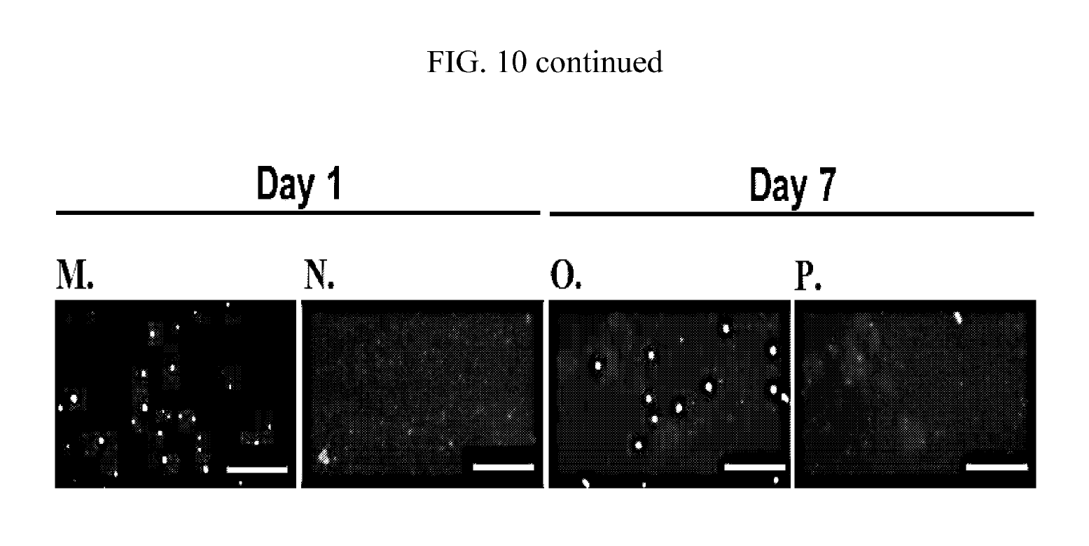

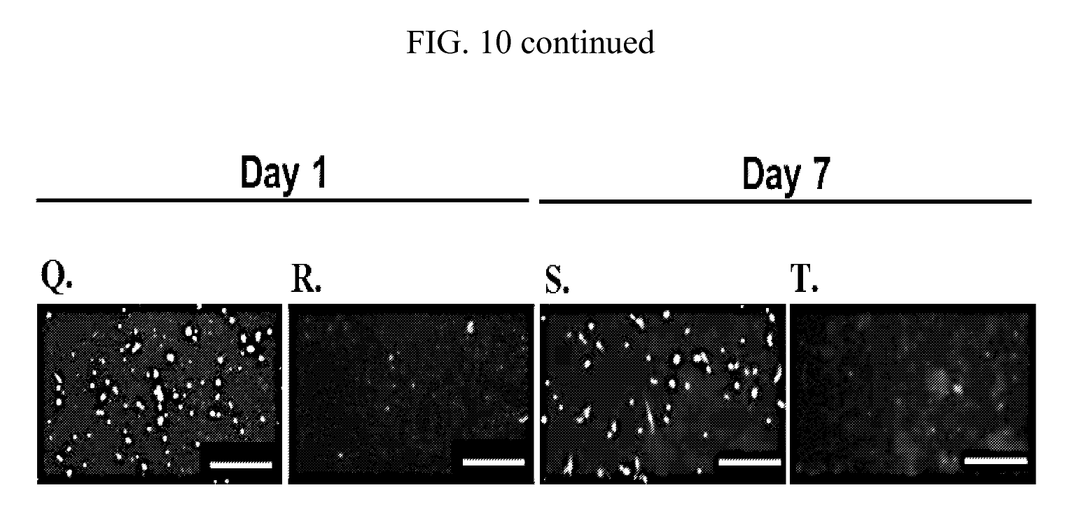

Cell viability studies were conducted using a Live/Dead Viability Kit (Invitrogen, Molecular Probes) using 2.0 μM calcein AM and 4.0 μM ethidium homodimer-1 (EthD-1). 200 μl of the combined Live/Dead assay reagent was added to the cells seeded onto IPM-based biomaterial matrix cell scaffolds at day 1 and at day 7 (n=3). The plates were incubated for 30 minutes at room temperature in the dark. Following incubation, the cells were observed using a fluorescence inverted microscope (Olympus, DP70), FIG. 10A-T.

Cell Morphology:

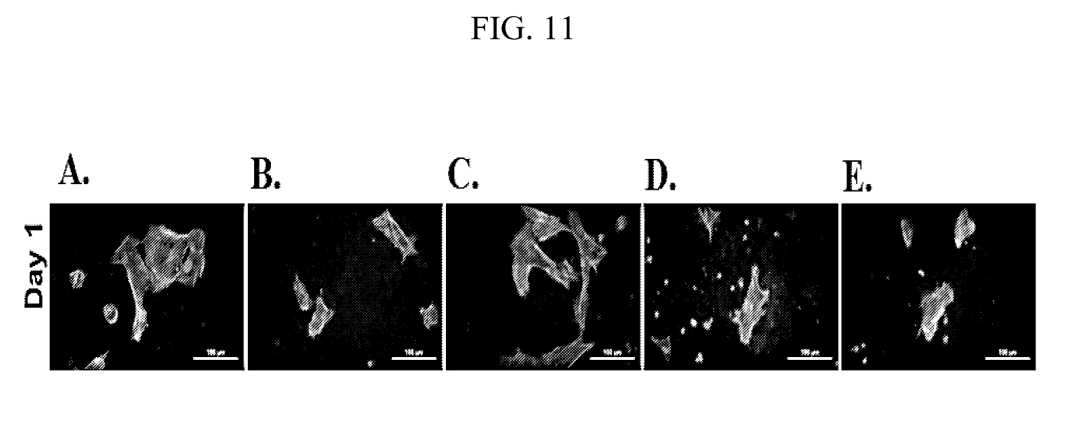

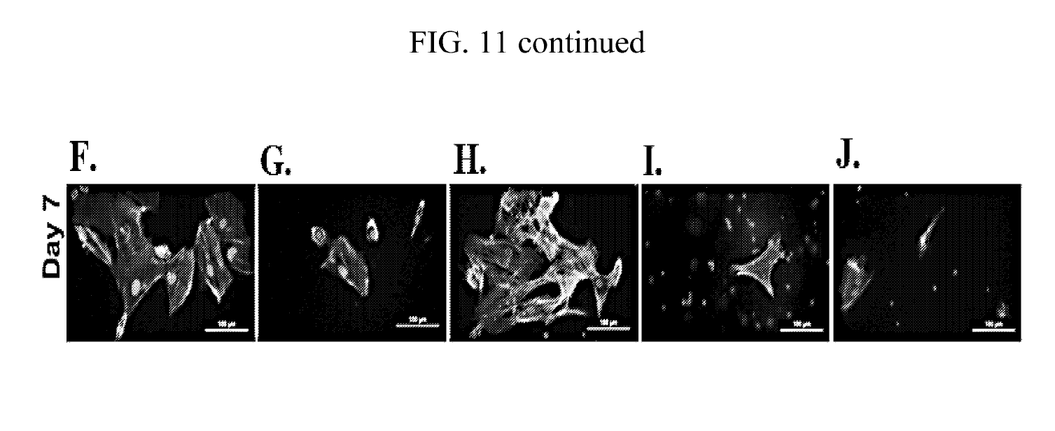

The cells on the IPM-based biomaterial cell scaffolds were fixed in 3.7% formaldehyde for 10 minutes at room temperature, then washed twice with PBS and permeabilized by 0.1% Triton X-100 (Sigma-Aldrich) in PBS for 5 minutes. After two washes with PBS, the actin structures of cells on the scaffold were labeled using phalloidin (Molecular Probes). Cells were incubated with 0.16 μM phalloidin in 1% BSA/PBS for 20 min at room temperature. Cells were counterstained with 5 ng/ml Hoechst 33258 (Molecular Probes) in PBS for 20 minutes at room temperature. Cell cytoskeleton was observed using a fluorescence microscope (Olympus DP70), FIG. 11A-J.

Cell Proliferation:

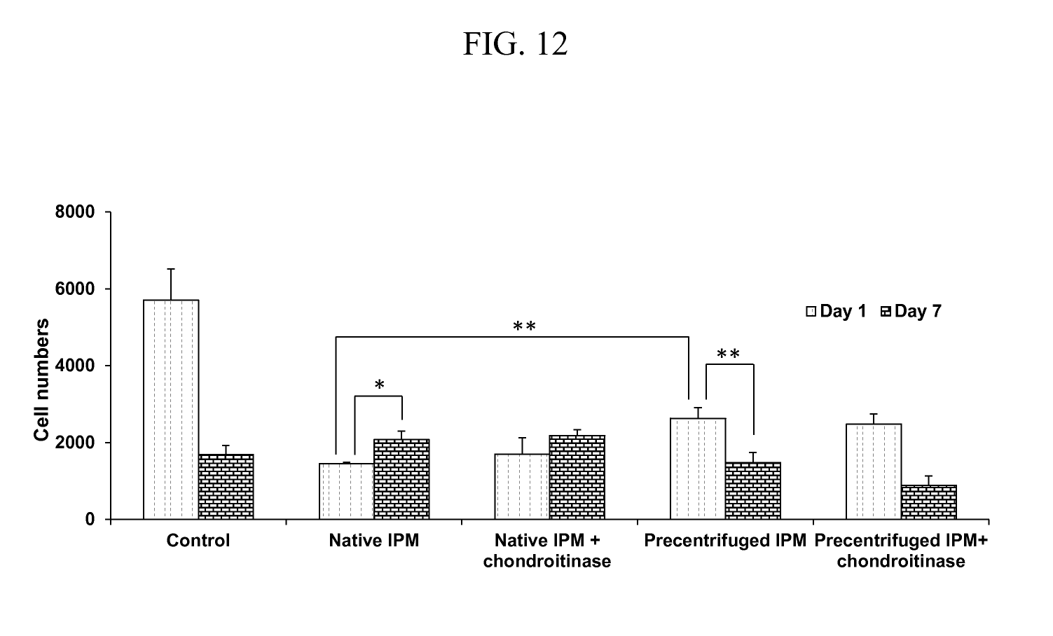

The cells on the IPM-based biomaterial cell scaffolds were for counted at day 1 and day 7 day (n=3) using the cyQUANT cell proliferation assay (Invitrogen), FIG. 12.

Immunocytochemistry:

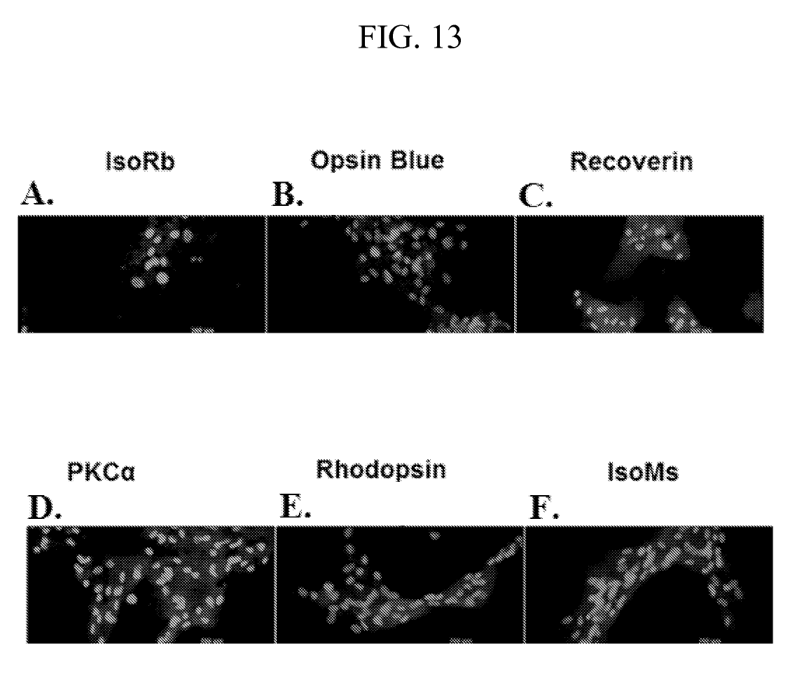

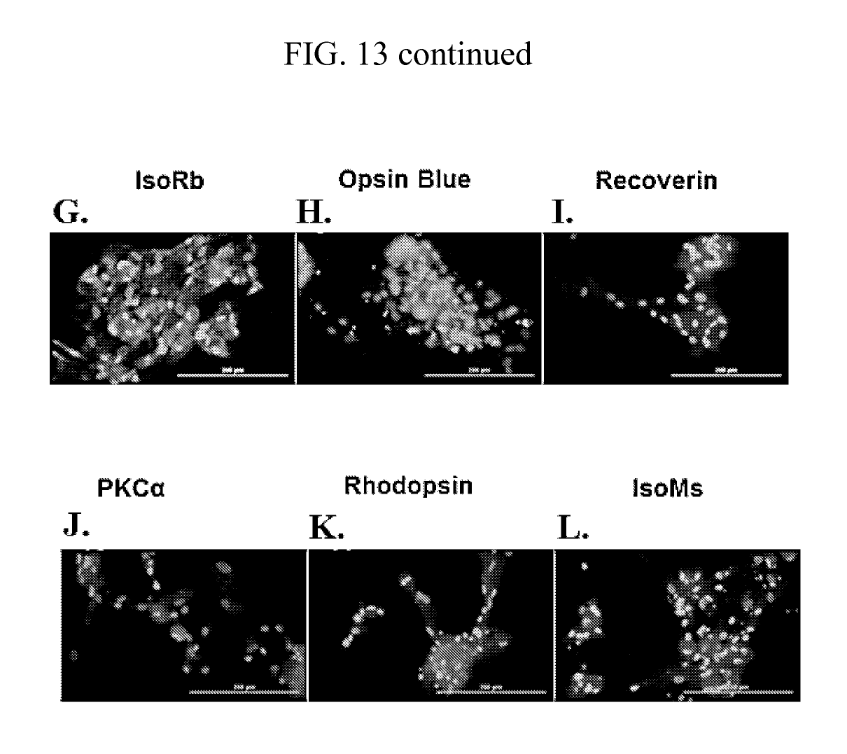

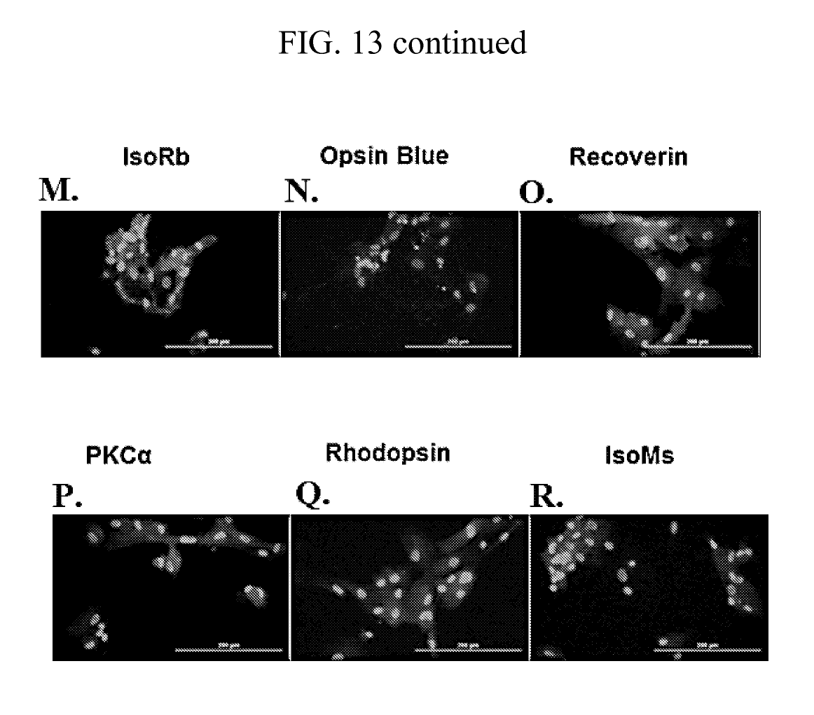

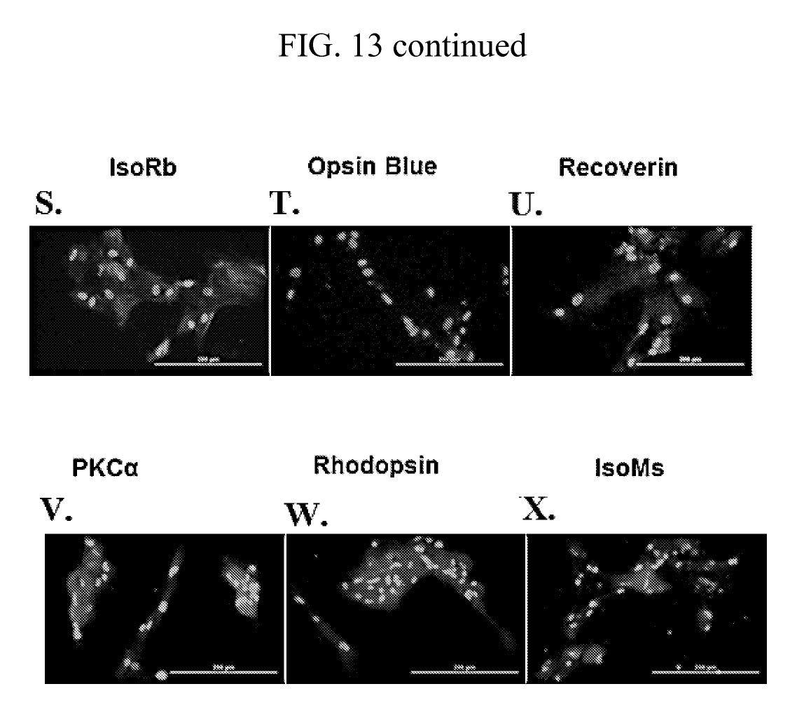

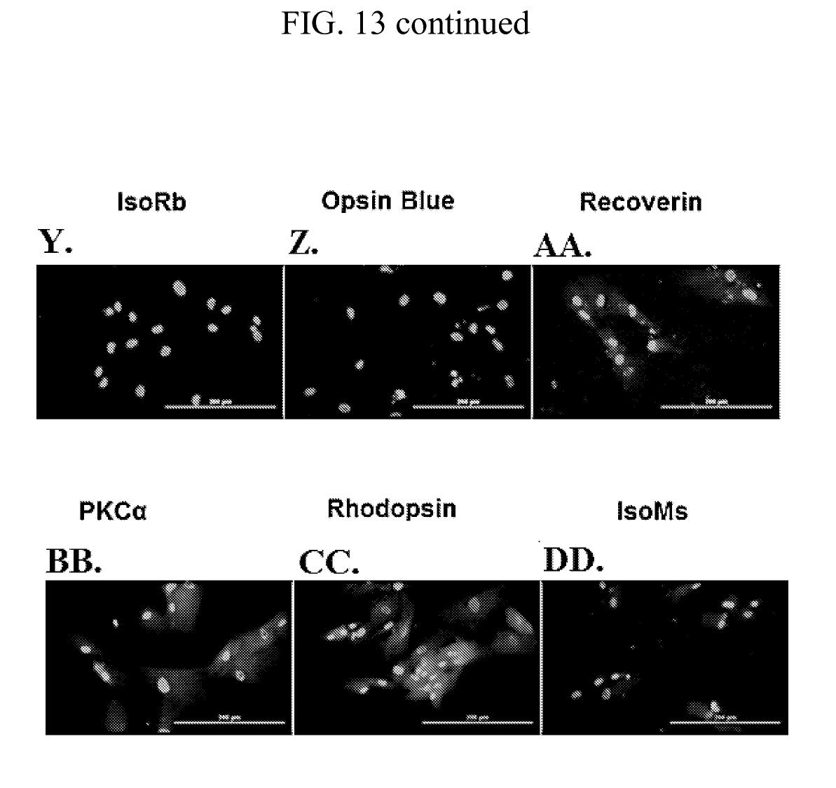

Cells were assessed via immunocytochemical analysis for expression of photoreceptor markers: opsin blue, recoverin, rhodopsin, PKC α and vimentin. For the immunocytochemistry assay, 4,000 hRPCs were plated in each well of 96 well culture plates (Nunc) containing tissue culture plate surface (control), and one of the following IPM-based biomaterial compositions: native IPM, native IPM treated with chondroitinase, pre-centrifuged IPM, and pre-centrifuged IPM treated with chondroitinase (n=3 for each IPM-based biomaterial matrix). After 1 day and 7 days of incubation, cells were washed in PBS, fixed (cold, freshly prepared 4% PFA), permeabilized (0.02% Triton X-100 in 5% BSA), blocked, and stained with primary antibodies overnight at 4° C. and probed with secondary antibodies (1:50, Goat Cy3-conjugated anti-rabbit or anti-mouse; Jackson Immunoresearch) for 1 hour at room temperature, FIG. 13.

Statistical Analysis:

All the experimental data in this example was expressed as mean±standard deviation. Experiments were repeated three times with three separate IPM preparations. All assay measurements were performed in triplicates on samples from three separate measurements. The significance was determined by the ANOVA test using MiniTab 15 software. The value of p<0.05 is considered as significant differences.

Results

A majority of the hRPCs seeded onto the IPM-based biomaterial cell scaffolds were viable, FIG. 10A-T. Cells at 7 days, FIGS. 10C, 10G, 10K, 10O, and 10S, all show significant cell growth as compare to live cell present at day 1, FIGS. 10A, 10E, 10I, 10M, and 10Q. Cells at 7 days showed low amounts of dead cells, FIGS. 10D, 10H, 10L, 10P, and 10T.

Cell viability was slightly reduced on the pre-centrifuged IPM-based biomaterial matrix cell scaffolds, FIG. 10M-P. Cell survival rate was higher on chondroitinase treated surfaces compared to untreated surfaces, FIGS. 10I-L and Q-T. These results showed that a majority of seeded hRPCs will survive on the native IPM-based biomaterial cell scaffolds.

A majority of the hRPCs on the IPM-based biomaterial cell scaffolds had an elongated and spindle shaped cell morphology, FIGS. 11B-E and 11G-J, whereas in the control, cells were spreading and fibroblastic in shape, FIGS. 11A and 11F. The native IPM-based biomaterial matrix cell scaffold had improved cell attachment and stratification as compared to the pre-centrifuged IPM-based biomaterial cell scaffold.

There was a greater number of hRPCs on pre-centrifuged IPM-based biomaterial cell scaffolds (2630±274) as compared to native IPM-based biomaterial cell scaffolds (1448±40) after 1 day of culture, FIG. 12. The chondroitinase treatment of the IPM-based biomaterial cell scaffolds did not make a substantial difference in cell proliferation. After day 7 of culture, the number of hRPCs on the pre-centrifuged IPM decreased significantly (1479±266), whereas the number of hRPCs increased on the native IPM-based biomaterial cell scaffolds (2072±221), FIG. 12. These results indicate that the native IPM-based biomaterial cell scaffolds possesses requisite cell survival and proliferative attributes, which are lost over time with pre-centrifuged IPM-based biomaterial cell scaffolds.

Protein expression and markers for cell differentiation on different IPM-based biomaterial cell scaffolds were analyzed by immunocytochemistry. Expression of Opsin Blue, recoverin, and rhodopsin were observed, FIG. 13. Expression of Opsin Blue indicated the development of cone cells. Expression of rhodopsin indicated the development of rod cells, i.e., photoreceptor cells. Up-regulation for PKCα indicated expression for bipolar cell development, FIG. 14. No or minimal expression of vimentin, a glial cell marker, was observed (data not shown). The results show that IPM-based biomaterial cell scaffolds induce differentiation of hRPCs in vitro.

These results show that the ocular biomaterial compositions of the present technology will support cell growth and differentiation. Accordingly, the ocular biomaterial compositions are useful as a cell delivery matrix for implantation of cells into the eye prevent or treat degenerative eye diseases or disorders.

### Example 10

### RPCs Seeded in IPM-Based Biomaterial Cell Delivery Matrix Restore Vision

This example will show that retinal progenitor cells (RPCs) delivery by an IPM-based biomaterial cell delivery matrix restores vision in mice with a retinitis pigmentosa (RP) phenotype.

Methods

Transplantation surgeries are performed on rhodopsin knockout mouse (Rho−/−) mice. The Rho−/− mouse displays an RP-like phenotype, with an age-dependent decline in rod number and function. The Rho−/− mouse is generated by a replacement mutation in exon 2 of the rhodopsin gene in the C57Bl/6J strain, the mice show reduced outer nuclear layer (ONL) thickness and the absence of a rod ERG response at 48 days of age, with virtually complete rod photoreceptor loss by 3 months.

Transplantation Surgery:

Mice are placed under general anesthesia, the pupil is dilated, and topical anesthetic is applied to the eye. The eyelid is retracted and the globe is stabilized with sutures, and an incision (0.5-1.0 mm) is made in the lateral posterior sclera. Murine RPCs seeded in IPM-based biomaterial cell delivery matrix is inserted into the subretinal space through the sclerotomy using an injection apparatus. Ten C57BL/6 wild-type mice and ten Rho−/− mouse are treated with the RPCs IPM-based biomaterial cell delivery matrix. Ten Rho−/− mice are implanted with RPCs only. Ten Rho−/− mice are implanted with IPM-based biomaterial composition only (i.e., no RPCs). Ten Rho−/− mice will not have implantations. Each mouse that receives a transplant receives the transplant in a single eye. The scleral incision is closed and all other sutures removed, the transplants remain in the subretinal space for 1 month.

Testing of Retinal Function:

Electroretinography (ERG) is performed on the mice at 3 weeks post-subretinal transplantation. Mice are dark-adapted for 12 hours, are anesthetized, their pupils are dilated, and the mice are placed on a heated recording stage maintained at 37° C. Gold ring electrodes are placed directly onto the corneal surface of eyes pre-coated with a 2.5% hydroxypropylmethylcellulose solution (Gonak, Akorn). Responses to 5 test flashes are recorded for each mouse and all ERGs are carried out under scotopic conditions. ERG signals are amplified 10,000×, filtered between 1 Hz to 3 kHz, and sampled at 5 kHz.

Analysis of Transplanted Tissue:

Mice are sacrificed and eyes containing transplants are enucleated, affixed in 4% paraformaldehyde, rinsed in buffer, cryoprotected, frozen, and sectioned Immunohistochemistry and fluorescence microscopy are used to evaluate survival, integration into host tissue, and differentiation of transplanted cells as described above for retinal explants cultures.

Results

It is anticipated that Rho−/− mice implanted with RPCs only and RPCs seeded in an IPM-based biomaterial composition will display higher retinal function are compared to non-implanted Rho−/− mice and IPM-based biomaterial only Rho−/− mice.

It is anticipated that Rho−/− mice implanted with RPCs only and RPCs seeded in an IPM-based biomaterial composition will display similar retinal function as compared to the C57BL/6 wild-type mice implanted with RPCs seeded in an IPM-based biomaterial composition.

It is anticipated that Rho−/− mice implanted with RPCs seeded in an IPM-based biomaterial composition will display higher retinal function as compared to Rho−/− mice implanted with RPCs only.

It is anticipated that tissue analysis will show that Rho−/− mice implanted with RPCs seeded in an IPM-based biomaterial composition will display a greater number of RPCs that migrated and integrated into the retina, will have a greater number of surviving RPCs, and will have a greater number of differentiated cell as compared to Rho−/− mice implanted with RPCs only.

These results will show that cells seeded in an ocular biomaterial composition improve cell survival, cell proliferation, cell integration, and cell differentiation when implanted into the eye. These results will show that the ocular biomaterial compositions disclosed herein are useful for the prevention and treatment of degenerative eye diseases or disorders.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into sub-ranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A composition comprising a decellularized interphotoreceptor matrix (IPM)-based biomaterial.

2. The composition of claim 1, further comprising a decellularized retina-based biomaterial.

3. The composition of claim 1, wherein the composition is substantially free of one or more biological inhibitory compounds selected from the group consisting of: chondroitin sulfate, proteoglycans, and leukocyte antigens.

4. The composition of claim 1, further comprising one or more biological agents, synthetic agents, or a combination thereof, wherein the biological agent and/or synthetic agent is dispersed throughout the decellularized IPM-based biomaterial.

5. The composition of claim 4, wherein the biological agent is one or more selected from the group consisting of: collagen, fibronectin, and transglutaminase.

6. The composition of claim 4, wherein the synthetic agent is one or more selected from the group consisting of polymers, poly(lactic-co-glycolic acid) (PLGA), polyglycerol sebacate (PGS), poly(L-lactic acid) (PLLA), poly(methylmethacrylate) (PMMA), and polycaprolactone (PCL).

7. The composition of claim 1, further comprising one or more additional components, wherein the additional components are selected from the group consisting of laminin, an antibiotic, an antiviral, an antifungal, growth factors, cytokines/chemokines, hormones, cell signaling molecules, a gelling agent, a protease inhibitor, cell media, and bovine serum proteins.

8. The composition of claim 1, wherein the decellularized IPM-based biomaterial is derived from a mammal, an amphibian, a fish, or a combination thereof.

9. The composition of claim 8, wherein the decellularized IPM-based biomaterial is derived from a species in which retinal regeneration occurs.

10. The composition of claim 1, further comprising retinal progenitor epithelium cells (RPCs).

11. The composition of claim 10, wherein the RPCs are human.

12. The composition of claim 1, wherein the composition is applied onto a substrate, wherein the substrate is selected from the group consisting of a biological or polymer membrane, tissue culture inserts, and cell culture plates.

13. The composition of claim 4, wherein the composition is formed as a cell scaffold.

14. A method for preventing or treating retinal degeneration in a subject in need thereof comprising: combining retinal cells and/or retinal progenitor cells (RPCs) with the composition of claim 1 to form a cell delivery matrix; and implanting the cell delivery matrix in at least one eye of the subject.

15. The method of claim 14, wherein the retinal degeneration is caused by a disease or disorder selected from the group consisting of: macular degeneration, retinitis pigmentosa, diabetic retinopathy, hypertensive retinopathy, radiation retinopathy, solar retinopathy, and retinopathy of prematurity.

16. The method of claim 14, wherein the cell delivery matrix is implanted in the subretinal space of the eye.

17. The method of claim 14, wherein the subject is human.

18. A method for restoring vision to a subject in need thereof, the method comprising: combining retinal cells and/or retinal progenitor cells (RPCs) with the composition of claim 1 to form a cell delivery matrix; and implanting the cell delivery matrix in at least one eye of the subject.

19. The method of claim 18, wherein the cell delivery matrix is implanted in the subretinal space of the eye.

20. The method of claim 18, wherein the subject suffers from a disease or disorder selected from the group consisting of: macular degeneration, retinitis pigmentosa, diabetic retinopathy, hypertensive retinopathy, radiation retinopathy, solar retinopathy, and retinopathy of prematurity.
